(19) United States
(12) Patent Application Publication
(10) Pub. No.: US 2006/0079497 A1
(43) Pub. Date: Apr. 13, 2006

(54) Lavendamycin analogues and methods of synthesizing and using lavendamycin analogues

(75) Inventors: Mohammad Behforouz, Muncie, IN (US); Nancy C. Behforouz, Muncie, IN (US)

Correspondence Address: BRINKS HOFER GILSON & LIONE, P.O. BOX 10395, CHICAGO, IL, 60610, US

(73) Assignee: Ball State University

(21) Appl. No.: 10/964,450

(22) Filed: Oct. 12, 2004

(51) Int. Cl. A61K 31/4745; C07D 471/02

(52) U.S. Cl. 514/210.21; 546/85; 514/291

(57) ABSTRACT

Lavendamycin analogues, methods for their synthesis, and methods for their use in the treatment of diseases such as cancer and HIV infection are described.

### FEDERALLY SPONSORED RESEARCH OR DEVELOPMENT

This invention was made in part with Government support (Grant No. R01 CA74245 awarded by the National Institutes of Health, and Grant No. DHP110 awarded by the American Cancer Society). The Government may have certain rights in this invention.

### FIELD OF THE INVENTION

The present invention relates to lavendamycin analogues, to methods of their preparation, and to methods for their use in the treatment of illnesses such as cancer and HIV infection.

### BACKGROUND

Human immunodeficiency virus (HIV) infection, with its clinical progression to AIDS, is one of the leading causes of morbidity and mortality in the world. Although highly active anti-retroviral therapy (HAART) using a combination of anti-retrovirals has been effective in suppressing HIV load and decreasing mortality of these infections, the emergence of drug resistance among HIV and inherent toxicity of most of the anti-retroviral therapies makes the continued search for novel anti-HIV drugs imperative. Indeed, it is now estimated that over 30% of patients are failing combination therapy. Such failure is partly due to the development of drug resistance and drug toxicity but also to the lack of patient compliance due to demanding drug dosing.

Currently approved HIV drugs includes six nucleoside HIV reverse transcriptase inhibitors, zidovudine (AZT), didanosine (ddI), stuvudine (d4T), lamivudine, (3TC), zalcitabine (ddC) and abacavir (ABC); three normucleoside RT inhibitors, nevirapinne, delavirdine and efavirenz; and five protease inhibitors, squinavir, indinavir, ritonavir, nelfinavir, amprenovir and lopiavir. Currently, HAART using different combinations of these drugs is the preferred treatment for AIDS patients. Non-nucleoside HIV-1 reverse transcriptase inhibitors (NNRTI) are becoming increasingly important additions to this combination retroviral therapy and these compounds tend to have good anti-viral potency, high specificity and low toxicity. It has been suggested that NNRTIs used in combination regimens with protease inhibitors (PI) may be used in the future either as first-line or second-line or salvage therapy in patients who need to change anti-retroviral treatments. Furthermore, anti-HIV combination strategies that demonstrate favorable drug interactions (e.g., synergy) may allow the use of individual agents below their toxic concentrations, provide more complete viral suppression, and limit the emergence of drug-resistant HIV mutant.

Earlier work has shown that two structurally related antibiotics produced by Streptomyces, streptonigrin (1) and lavendamycin (2a), have significant biological activity across a broad spectrum including antitumor, antibacterial, and antiviral activity.

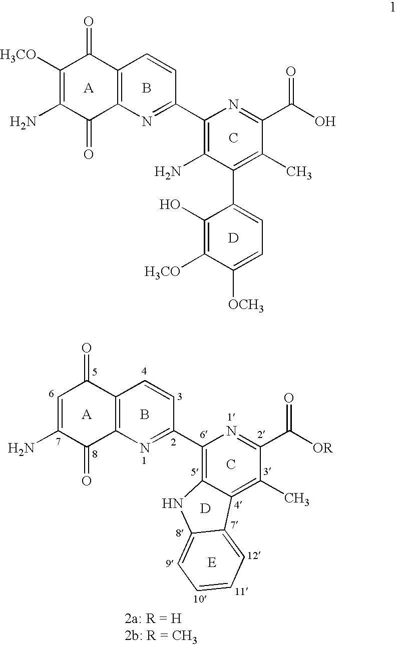

Streptonigrin and several of its derivatives, particularly its alkyl esters, were shown to have potent anti-reverse transcriptase activity against HIV-RT. Unfortunately, both the streptonigrins and the parent lavendamycin were found to be highly toxic. Until recently, evaluation of the therapeutic potential of synthetic lavendamycin analogs was precluded by a lack of efficient synthetic methods. Indeed, the first reported total synthesis of lavendamycin methyl ester (2b) involved a large number of steps with low overall yields of 0.5-2%. In 1993 and 1996, we reported short and efficient methods for the synthesis of lavendamycin methyl ester (2b) in excellent overall yields of 33-40% (e.g., see: Behforouz, M.; Gu, Z.; Cai, W.; Horn, M. A.; Ahmadian, M. J. Org. Chem. 1993, 58, 7089; and Behforouz, M.; Haddad, J.; Cai, W.; Arnold, M. B.; Mohammadi, F.; Sousa, A. C.; Horn, M. A. J. Org. Chem. 1996, 61, 6552).

In view of the significant biological activities of streptonigrin and lavendamycin, there is a pressing need for the development of new less toxic analogues that may be efficiently synthesized and used in the treatment of illnesses such as cancer and HIV infection.

### SUMMARY

The scope of the present invention is defined solely by the appended claims, and is not affected to any degree by the statements within this summary.

By way of introduction, a compound embodying features of the present invention has a structure: 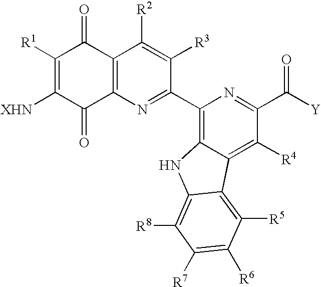
or a pharmaceutically acceptable salt thereof, wherein:

X is hydrogen or R¹OC(O)—;

Y is hydroxy, alkoxy, amino, Ph(CH₂)ₘO—, HO(CH₂)ₙO— or —O(CH₂)ₒOPO₃H₂, wherein m, n, and o are each independently 1, 2, 3, 4, 5, 6, 7, 8, 9 or 10;

R¹ is hydrogen, amino, alkoxy, halo, acyl, aziridinyl, pyrrolidinyl, piperidinyl or pyridinyl;

R², R³, R⁴, R⁵, R⁶, R⁷, and R⁸ are each independently hydrogen or a substituted or unsubstituted radical selected from the group consisting of alkyl, aryl, cycloalkyl, alkenyl, alkynyl, heteroalkyl, heterocyclic, heteroalkenyl, heteroalkynyl, halo, nitro, cyano, alkoxy, amino, amido, thioamido, acyl, thioacyl, and imino; and

R¹⁰ is hydrogen or a substituted or unsubstituted radical selected from the group consisting of alkyl, aryl, cycloalkyl, alkenyl, alkynyl, heteroalkyl, heterocyclic, heteroalkenyl, and heteroalkynyl;

with a proviso that when R¹ is hydrogen, halo, amino, alkoxy or acyl, then Y is not hydroxy, alkoxy or amino.

A method of treating cancer embodying features of the present invention includes administering to an animal in need thereof a therapeutically effective amount of a compound of a type described above.

A method of treating HIV infection embodying features of the present invention includes administering to an animal in need thereof a therapeutically effective amount of a compound of a type described above.

A method embodying features of the present invention for synthesizing a compound having a formula: 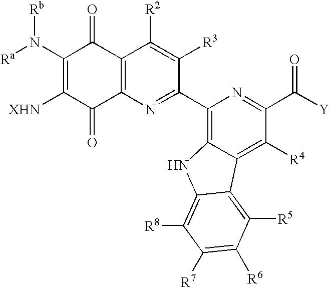
includes reacting a compound having a structure: 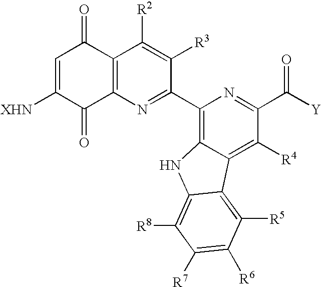
with a compound having a structure: 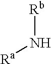
wherein:

X is hydrogen or R¹⁰C(O)—;

Y is hydroxy, alkoxy or amino;

R^{a} and R^{b} are either (a) independently hydrogen or a substituted or unsubstituted radical selected from the group consisting of alkyl, aryl, cycloalkyl, alkenyl, alkynyl, heteroalkyl, heterocyclic, heteroalkenyl, and heteroalkynyl or (b) together with the nitrogen atom to which they are attached form a three-, four-, five-, six-, seven- or eight-membered cyclic ring;

R¹ is amino, aziridinyl, pyrrolidinyl, piperidinyl or pyridinyl;

R², R³, R⁴, R⁵, R⁶, R⁷, and R⁸ are each independently hydrogen or a substituted or unsubstituted radical selected from the group consisting of alkyl, aryl, cycloalkyl, alkenyl, alkynyl, heteroalkyl, heterocyclic, heteroalkenyl, heteroalkynyl, halo, nitro, cyano, alkoxy, amino, amido, thioamido, acyl, thioacyl, and imino; and

R¹⁰ is hydrogen or a substituted or unsubstituted radical selected from the group consisting of alkyl, aryl, cycloalkyl, alkenyl, alkynyl, heteroalkyl, heterocyclic, heteroalkenyl, and heteroalkynyl.

### BRIEF DESCRIPTION OF THE DRAWINGS

FIG. 1 shows a graph of the National Cancer Institute (NCI) in vitro screening data for lavendamycin analogue 14.

FIG. 2 shows a graph of the NCI in vitro screening data for lavendamycin analogue 17.

FIG. 3 shows a graph of the NCI in vitro screening data for lavendamycin analogue 19.

FIG. 4 shows a graph of the NCI in vitro screening data for lavendamycin analogue 21.

FIG. 5 shows a graph of the NCI in vitro screening data for a lavendamycin analogue embodying features of the present invention.

FIG. 6 shows a graph of the NCI in vitro screening data for a lavendamycin analogue embodying features of the present invention.

FIG. 7 shows a graph of the NCI in vitro screening data for a lavendamycin analogue embodying features of the present invention.

### DETAILED DESCRIPTION

Novel lavendamycin analogues have been discovered and are described hereinbelow, including but not limited to water-soluble lavendamycin analogues and lavendamycin analogues substituted at the C-6 position. In addition, short and practical methods for the synthesis of these lavendamycin analogues have been discovered and are described below.

Lavendamycin analogs embodying features of the present invention have antitumor and antimicrobial (e.g., antibacterial, antiviral and antiparasitic) activity. In accordance with the present invention, methods of treating animals having a tumor or suffering from a microbial infection are provided which comprise administering to the animals an effective amount of a lavendamycin analog embodying features of the present invention or a pharmaceutically-acceptable salt thereof. Also provided is the use of the above-described lavendamycin compounds in treating cancer. The present invention also provides pharmaceutical compositions comprising a lavendamycin analog, or a pharmaceutically-acceptable salt thereof, in combination with a pharmaceutically-acceptable carrier.

Lavendamycin analogs embodying features of the present invention also have anti-HIV Reverse Transcriptase (HIV-RT) activity by themselves as well as in combination with additional agents (e.g., 3′-azido-3′-deoxythymidine or AZT). Accordingly, the present invention provides for the use of lavendamycin analogs in treating HIV infection, and for methods and compositions for treating HIV infection with lavendamycin analogs and with combinations of lavendamycin analogs and agents such as AZT.

The present invention also provides methods of inhibiting the growth of microbes comprising contacting the microbe with a lavendamycin analog embodying features of the present invention, or a salt thereof. For example, lavendamycin analogs in accordance with the present invention may be added to liquids to inhibit microbial growth therein. The lavendamycin analogs may also be formulated into disinfectant preparations useful for inhibiting microbial growth on surfaces.

Throughout this description and in the appended claims, the following definitions are to be understood:

The term “alkyl” refers to a straight or branched chain alkyl residue containing from 1 to 20 carbon atoms. Representative alkyl residues include but are not limited to methyl, ethyl, n-propyl, iso-propyl, n-butyl, iso-butyl, sec-butyl, tert-butyl, n-pentyl, 2-methylbutyl, 2,2-dimethylpropyl, iso-amyl, n-hexyl, 2-methylpentyl, 2,2-dimethylbutyl, n-heptyl, 2-methylhexyl, n-octyl, and the like.

The term “aryl” refers to a residue comprising at least one aromatic ring of 5 or 6 carbon atoms. Representative aryl residues include but are not limited to phenyl, tolyl, biphenyl, naphthyl, cyclopentadienyl, and the like.

The term “cycloalkyl” refers to an aliphatic ring having from 3 to 8 carbon atoms. Representative cycloalkyl residues include but are not limited to cyclopropyl, cyclopentyl, cyclohexyl, and the like.

The term “alkenyl” refers to a straight or branched chain alkyl residue which contains from 1 to 20 carbon atoms and at least one carbon-carbon double bond. Representative alkenyl residues include but are not limited to vinyl, allyl, 1,1-dimethyl allyl, and the like.

The term “alkynyl” refers to a straight or branched chain alkyl residue which contains from 1 to 20 carbon atoms and at least one carbon-carbon triple bond. Representative alkynyl residues include but are not limited to ethynyl, propenyl, and the like.

The term “heteroalkyl” refers to an alkyl residue containing one or more heteroatoms, with representative heteroatoms including but not limited to oxygen, sulfur, and nitrogen.

The term “heterocyclic” refers to a cycloalkyl or aryl residue containing one or more heteroatoms, with representative heteroatoms including but not limited to oxygen, sulfur, and nitrogen. Representative heterocyclic residues include but are not limited to thienyl, furyl, pyrrolyl, imidazolyl, pyrazolyl, pyridyl, pyrazinyl, pyrimidinyl, pyridazinyl, oxazolyl, isothiazolyl, isoxazolyl, thiazolyl, oxadiazolyl, thiadiazolyl, aziridinyl, pyrrolidinyl, piperidinyl, and the like.

The term “heteroalkenyl” refers to an alkenyl residue containing one or more heteroatoms, with representative heteroatoms including but not limited to oxygen, sulfur, and nitrogen.

The term “heteroalkynyl” refers to an alkynyl containing one or more heteroatoms, with representative heteroatoms including but not limited to oxygen, sulfur, and nitrogen.

The term “amino” refers to amine functionalities containing all manner of substitution (e.g., primary, secondary, tertiary, and the like).

The above-described alkyl, aryl, cycloalkyl, alkenyl, alkynyl, heteroalkyl, heterocyclic, heteroalkenyl and heteroalkynyl residues may be unsubstituted or substituted with one or more substituents. Suitable substituents include but are not limited to R^{x}, NH₂, R^{x}NH, (R^{x})₂N, CN, N₃, NO₂, OH, halogen (Cl, Br, F, I), SH, R^{x}S, R^{x}SO₂, R^{x}SO, R^{x}O, COOH, COOR^{x}, COR^{x}, CHO, and CON(R^{x})₂, wherein R^{x} is an alkyl, cycloalkyl, aryl, alkenyl, alkynyl or heterocyclic residue.

A first series of lavendamycin analogues embodying features of the present invention have a general structure (A):

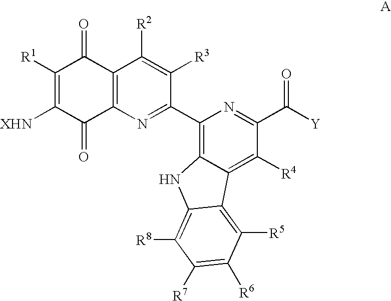

or a pharmaceutically acceptable salt thereof, wherein: X is hydrogen or R¹⁰C(O)—; Y is hydroxy, alkoxy, amino, Ph(CH₂)ₘO—, HO(CH₂)ₙO— or —O(CH₂)ₒOPO₃H₂, wherein m, n, and o are each independently 1, 2, 3, 4, 5, 6, 7, 8, 9 or 10; R¹ is hydrogen, amino, alkoxy, halo, acyl, or heterocyclic (e.g., aziridinyl, pyrrolidinyl, piperidinyl, pyridinyl or the like); R², R³, R⁴, R⁵, R⁶, R⁷, and R⁸ are each independently hydrogen or a substituted or unsubstituted radical selected from the group consisting of alkyl, aryl, cycloalkyl, alkenyl, alkynyl, heteroalkyl, heterocyclic, heteroalkenyl, heteroalkynyl, halo, nitro, cyano, alkoxy, amino, amido, thioamido, acyl, thioacyl, and imino; and R¹⁰ is hydrogen or a substituted or unsubstituted radical selected from the group consisting of alkyl, aryl, cycloalkyl, alkenyl, alkynyl, heteroalkyl, heterocyclic, heteroalkenyl, and heteroalkynyl. When R¹ is hydrogen, halo, amino, alkoxy or acyl, then Y is not hydroxy, alkoxy or amino.

A second series of lavendamycin analogues embodying features of the present invention have a general structure (B):

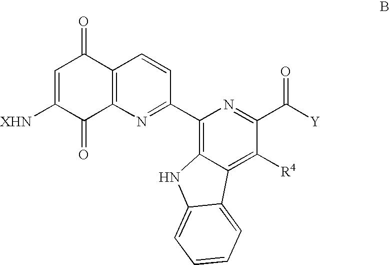

or a pharmaceutically acceptable salt thereof, wherein: X is hydrogen or R¹⁰C(O)—; Y is hydroxy, alkoxy, or amino; R⁴ is hydrogen, alkyl, aryl, cycloalkyl, alkenyl or alkynyl; and R¹⁰ is hydrogen or a substituted or unsubstituted radical selected from the group consisting of alkyl, aryl, cycloalkyl, alkenyl, alkynyl, heteroalkyl, heterocyclic, heteroalkenyl, and heteroalkynyl. In some embodiments, R¹⁰ is hydrogen, methyl, ethyl or n-propyl. In some embodiments, Y is —OH, —OCH₃, —O—CH₂CH₃, —O-n-C₄H₉, —O—n-C₈H₁₇, —NH₂, Ph(CH₂)ₘO—, HO(CH₂)ₙO— or —O(CH₂)ₒOPO₃H₂, wherein m, n, and o are each independently 1, 2, 3, 4, 5, 6, 7, 8, 9 or 10. In some embodiments, Y is —OCH₂Ph, HO(CH₂)₂O— or —O(CH₂)₂OPO₃H₂. In some embodiments, R⁴ is hydrogen or methyl.

The total synthesis of a number of novel demethyllavendamycin esters and amides and water-soluble lavendamycin derivatives via short and practical methods will now be described. Further description is provided in the article entitled “Novel Lavendamycin Analogues as Potent HIV-Reverse Transcriptase Inhibitors: Synthesis and Evaluation of Anti-Reverse Transcriptase Activity of Amide and Ester Analogues of Lavendamycin,” by Mohammad Behforouz et al. (J. Med. Chem., 2003, 46, 5773-5780), the entire contents of which are incorporated herein by reference, except that in the event of any inconsistent disclosure or definition from the present application, the disclosure or definition herein shall be deemed to prevail.

Syntheses of several novel lavendamycins possessing the full pentacyclic structure of the system and its C-7 amino and C-2′ acid functions and their derivatives have been discovered. In addition, efficient methods have been discovered to synthesize water-soluble lavendamycins useful for meaningful in vivo studies and potential chemotherapeutic application. As described below, these novel non-nucleoside lavendamycin agents have a high degree of activity in inhibiting HIV-RT. Moreover, these inhibitors also have low toxicity both in vitro and in vivo. In addition to being inhibitory alone, several of the analogs also display additive or synergistic activity against HIV RT together with AZT-triphosphate. The synthesis and biological evaluation of these lavendamycin analogs is also described.

Novel demethyllavendamycin esters and amides described below include water soluble derivatives, such as carboxylic acid 18 and dihydrogen phosphate 19 (Table 2), which were synthesized via short and efficient methods. Pictet-Spengler condensation of 7-N-acylamino-2-formylquinoline-5,8-diones with tryptophan esters or amide produced lavendamycins 11-17 (Table 1). Lavendamycins 18-21 were obtained respectively by further transformations of 13-15 and 17. Several of these lavendamycins were found to have potent anti-HIV reverse transcriptase with very low toxicity either in vitro or in vivo. These compounds also acted either additively or, in some cases, synergistically to inhibit enzyme activity together with AZT-triphosphate.

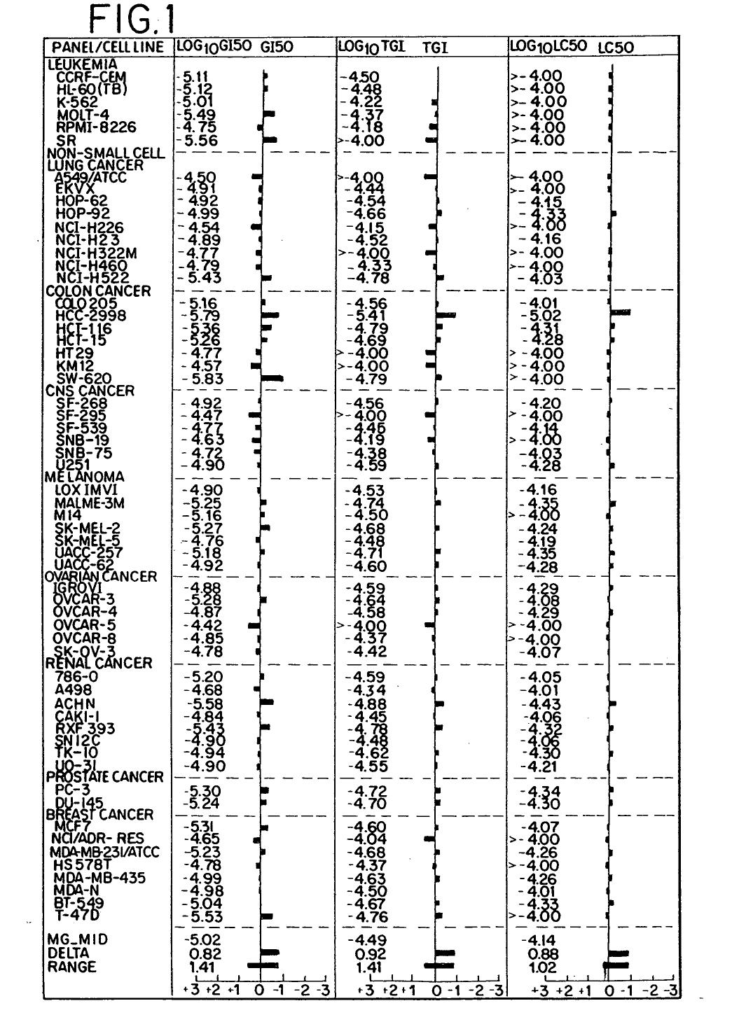

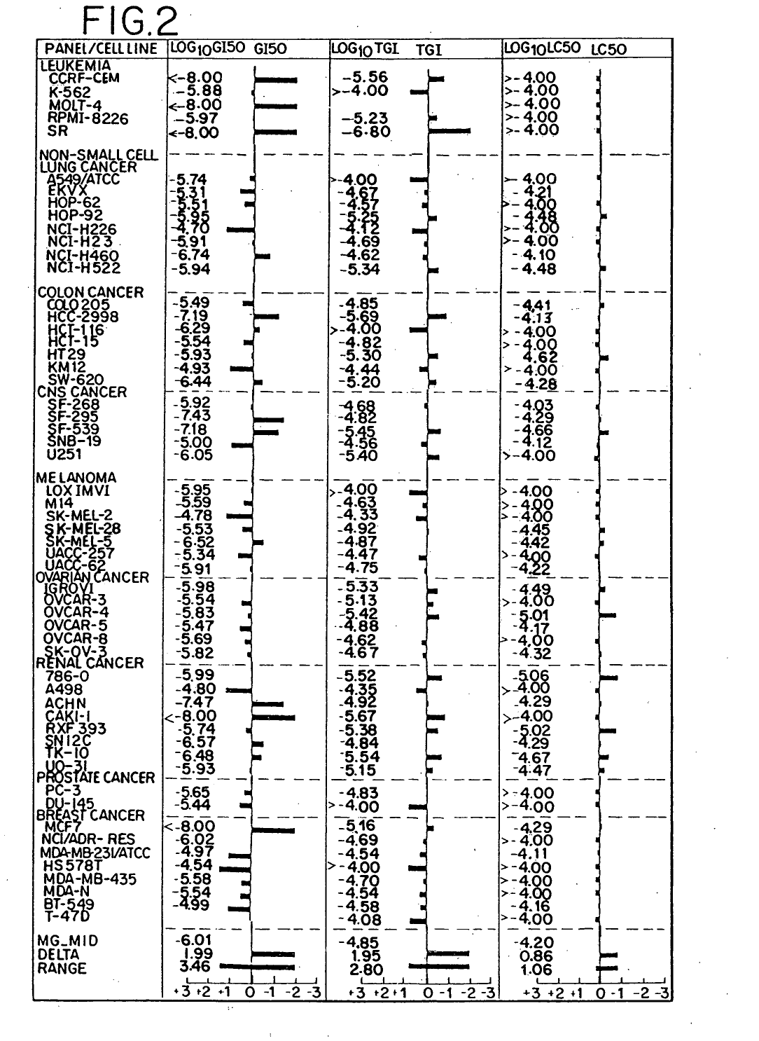

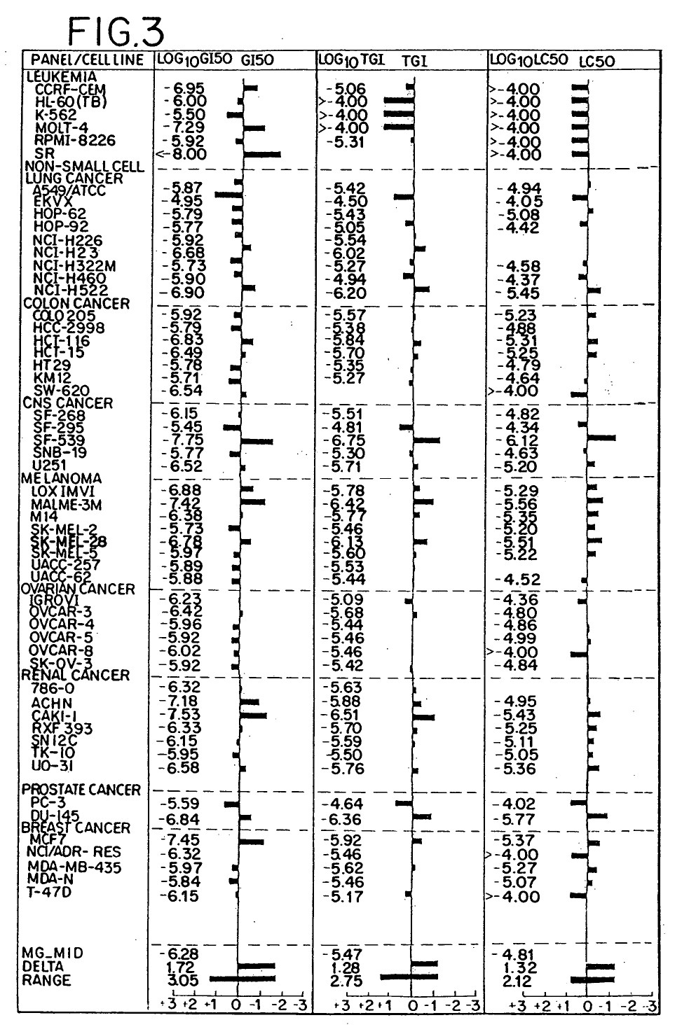

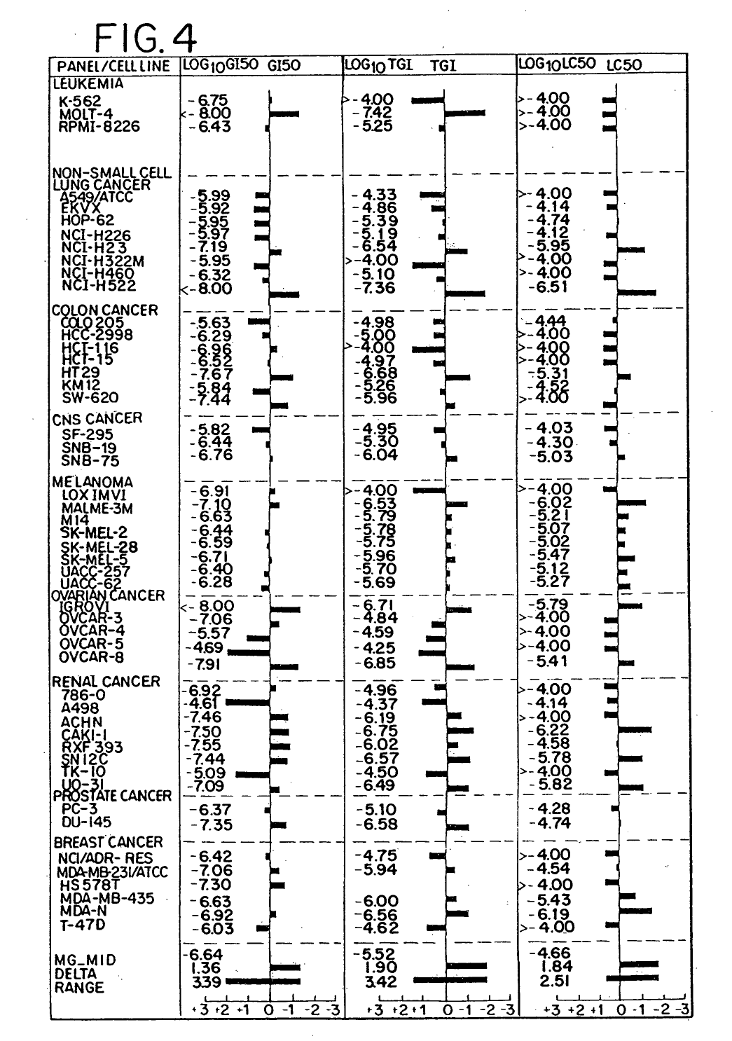

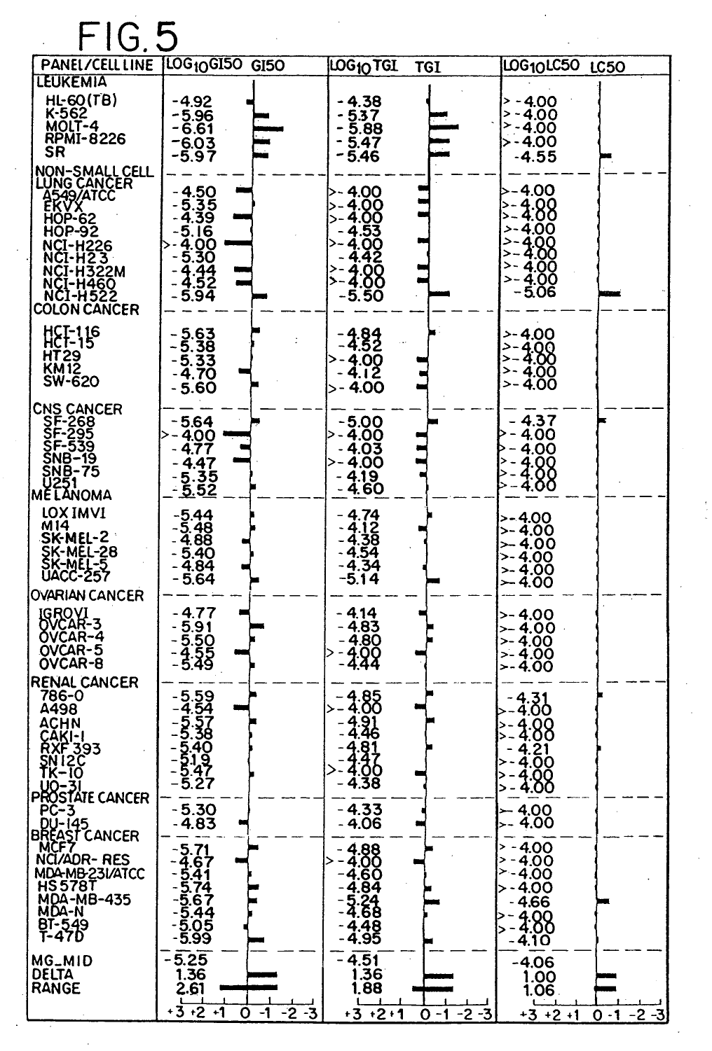

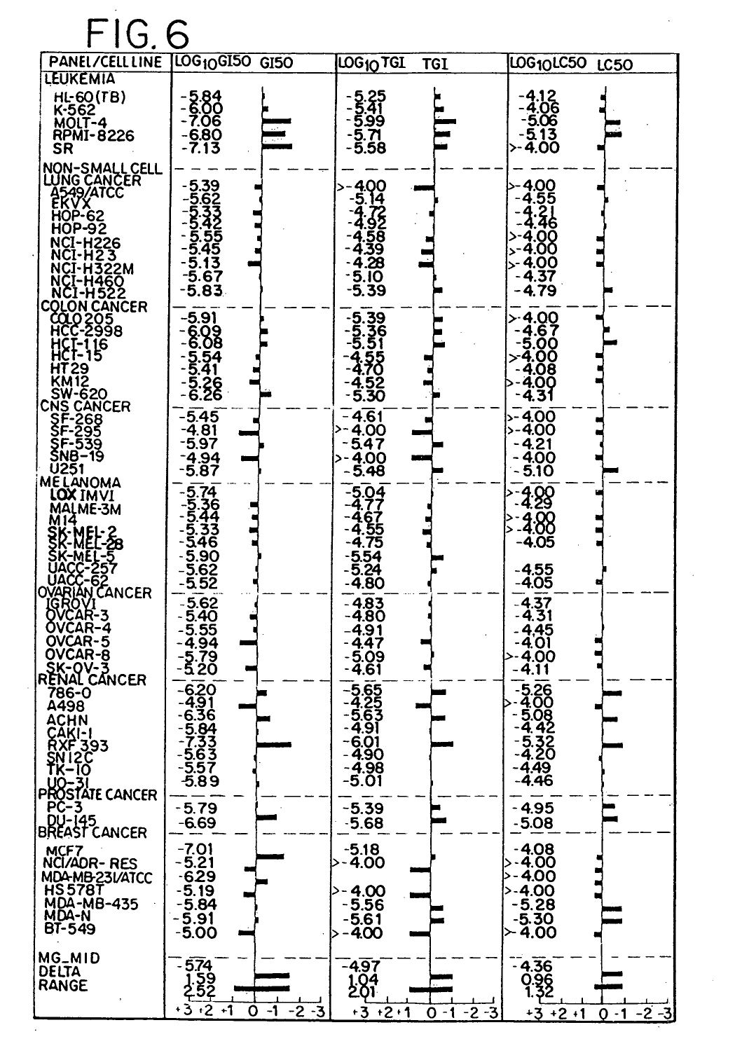

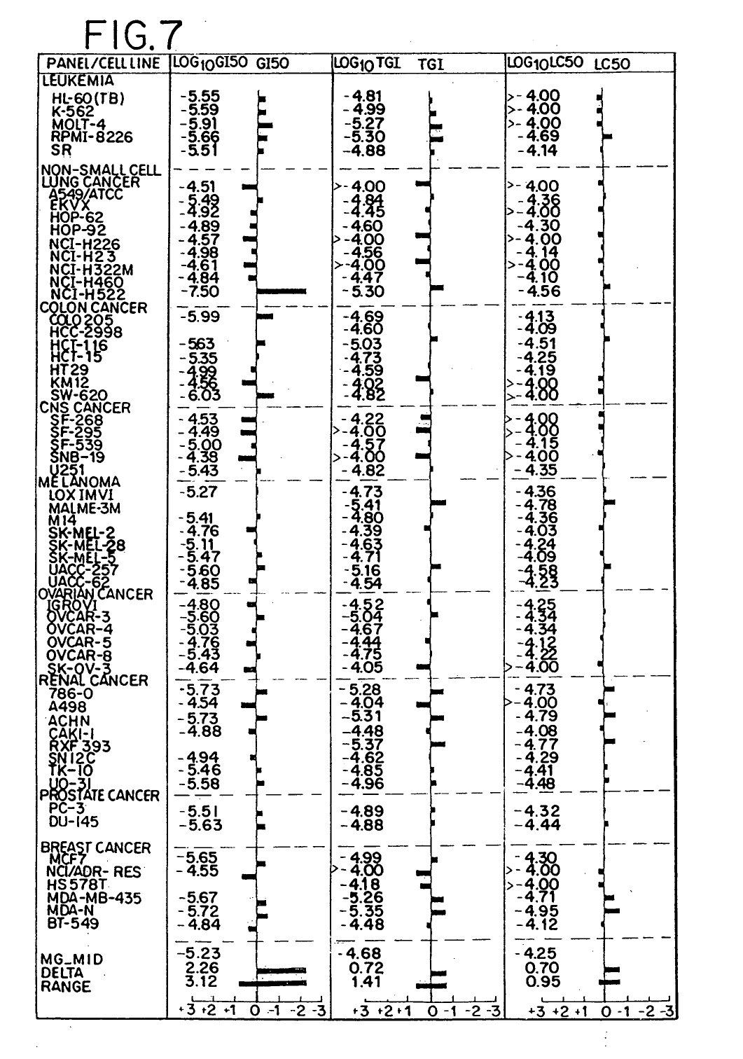

FIGS. 1-4 show graphs of the National Cancer Institute (NCI) in vitro screening data for lavendamycin analogues 14, 17, 19, and 21, respectively. FIGS. 5-7 show graphs of the NCI in vitro screening data for additional lavendamycin analogues embodying features of the present invention, which are similar to compounds 16 and 17. In FIG. 5, the lavendamycin analogue has a structure B as shown above in which X═—C(O)CH₃, Y═—NHCH₂CHOHCH₂OH, and R⁴═H. In FIG. 6, the lavendamycin analogue has a structure B as shown above in which X=—C(O)CH₂Cl, Y=NH₂, and R⁴═H. In FIG. 7, the lavendamycin analogue has a structure B as shown above in which X═—C(O)CH₃, Y═—NHCH₂CH₂OH, and R⁴═H.

Synthetic Chemistry

As shown in Scheme 1, Pictet-Spengler condensation of 7-N-acylamino-2-formylquinoline-5,8-diones 3 or 4 with β-methyltryptophan methyl ester (5) or tryptophan butyl, benzyl, 1,2-ethanediol, octyl, esters (6-9) or tryptophan amide (10) yielded the corresponding lavendamycin derivatives 11-17, respectively, in yields of 79, 54, 72, 94, 48, 62, and 63%.

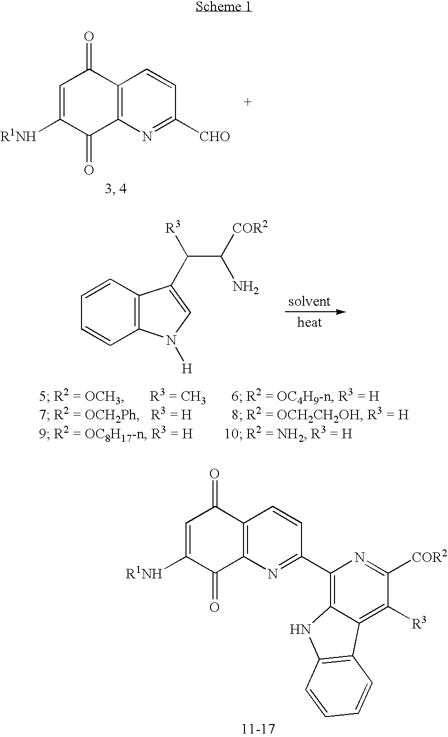

In a typical procedure, aldehydes 3 or 4 (0.1 mmol) were mixed with the corresponding tryptophan derivatives in 60 mL dry anisole or xylene under argon and while being magnetically stirred, the mixture was gradually heated to reflux over a period of 3 hours. The resulting clear solution was refluxed until TLC showed the absence of the starting materials. The mixture was concentrated or evaporated to dryness. The products were either precipitated from the concentrated solutions or purified by washing with acetone followed by ether. The structures of the resulting lavendamycins, the reaction conditions and the yields are shown in Table 1.

TABLE 1
Structures of Lavendamycins, Reactions Conditions and Yields

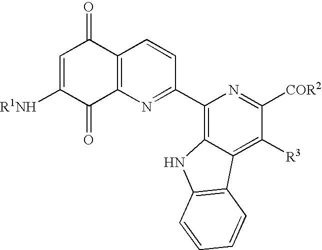

| No. | R¹ | R² | R³ | % yield | solvent | h (° C.) |
|---|---|---|---|---|---|---|
| 11 | CH₃CO | OCH₃ | CH₃ | 79 | xylene | 19 (reflux) |
| 12 | n-C₃H₇CO | OC₄H₉-n | H | 54 | anisole | 3.5 (reflux) |
| 13 | CH₃CO | OCH₂Ph | H | 72 | anisole | 18 (152) |
| 14 | CH₃CO | OCH₂CH₂OH | H | 87-94 | DMF/anisole | 2 (reflux) |
| 15 | CH₃CO | OC₈H₁₇-n | H | 48 | xylene | 3 (reflux) |
| 16 | CH₃CO | NH₂ | H | 62 | anisole | 14 (reflux) |
| 17 | n-C₃H₇CO | NH₂ | H | 63 | anisole | 13 (reflux) |

Carboxylic acid 18 was prepared in 62% yield by the hydrogenolysis of 13 in the presence of Pd/black in a CH₂Cl₂-MeOH-THF mixture. The free amino lavendamycins 20 and 21 were obtained by acid hydrolysis of the corresponding 15 and 16 respectively in 64 and 96.5% yields according to our reported method for the conversion of 11 to lavendamycin methyl ester 2b (e.g., see: Behforouz, M.; Gu, Z.; Cai, W.; Horn, M. A.; Ahmadian, M. “A Highly Concise Synthesis of Lavendamycin Methyl Ester,” J. Org. Chem., 1993, 58, 7089-7091; and Behforouz, M.; Haddad, J.; Cai, W; Arnold, M. B.; Mohammadi, F.; Sousa, A. C.; Horn, M. A. “Highly Efficient and Practical Syntheses of Lavendamycin Methyl Ester and Related Novel Quinolinediones,” J. Org. Chem., 1996, 61, 6552-6555). Table 2 shows the structures of compounds 18-21.

TABLE 2
Lavendamycins Derived from 13-15, 17

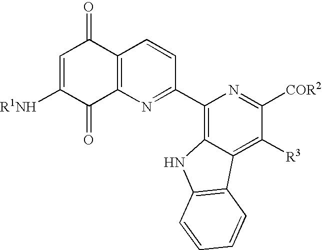

| | | | |
|---|---|---|---|
| No. | R¹ | R² | R³ |

| | | | |
|---|---|---|---|
| 18 | CH₃CO | OH | H |
| 19 | CH₃CO | OCH₂CH₂OPO₃H₂ | H |
| 20 | H | OC₈H₁₇-n | H |
| 21 | H | NH₂ | H |

Tryptophan 5 was prepared according to our own procedure (Behforouz, M.; Zarrinmayeh, H.; Ogle, M. E.; Riehle, T. J.; Bell, F. W. “β-Carbolines Derived from β-Methyltryptophan and a Stereoselective Synthesis of (2RS, 3SR)-β-Methyltryptophan Methyl Ester,” J. Heterocycl. Chem., 1988, 25, 1627-1632). Tryptophans 6, 7, 9, and 10 were obtained by the neutralization of the commercially available salts with ammonium hydroxide. The procedure for the preparation of 1,2-ethanediol ester 8 is shown in Scheme 2.

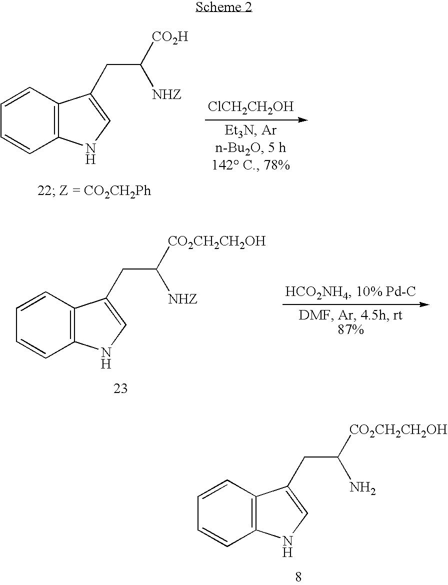

Aldehyde 3 was prepared according to our previously reported method (e.g., see: Behforouz, M.; Gu, Z.; Cal, W.; Horn, M. A., Ahmadian, M. “A Highly Concise Synthesis of Lavendamycin Methyl Ester,” J. Org. Chem., 1993, 58, 7089-7091; and Behforouz, M.; Haddad, J.; Cal, W.; Gu, Z. “Chemistry of Quinoline-5,8-diones,” J. Org. Chem., 1998, 63, 343-346) as shown in Scheme 3. A similar procedure (Scheme 3) was used for the preparation of 7-N-butyramido-2-formylquinoline-5,8-dione (4).

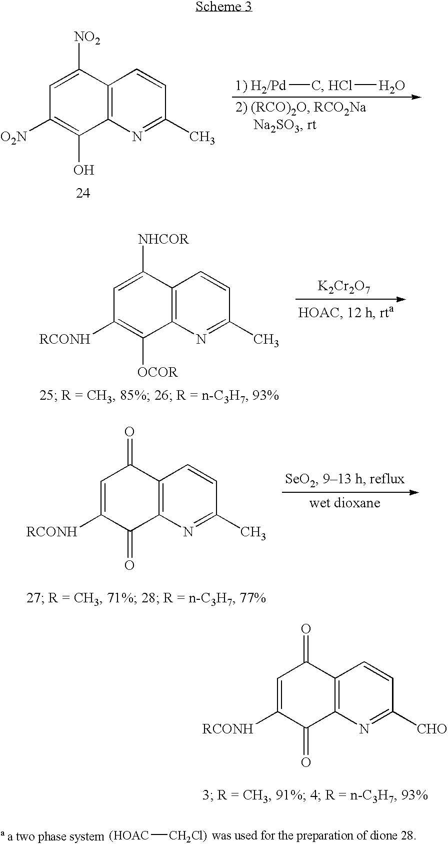

Water soluble dihydrogen phosphate 19 was prepared by the Mitsunobu method as shown in Scheme 4 (e.g., see: Mitsunobu, O.; Kato, K.; Kimura, J. “Selective Phosphorylation of 5-Hydroxy Groups of Thymidine and Uridine,” J. Amer. Chem. Soc., 1969, 91, 6510-6511). 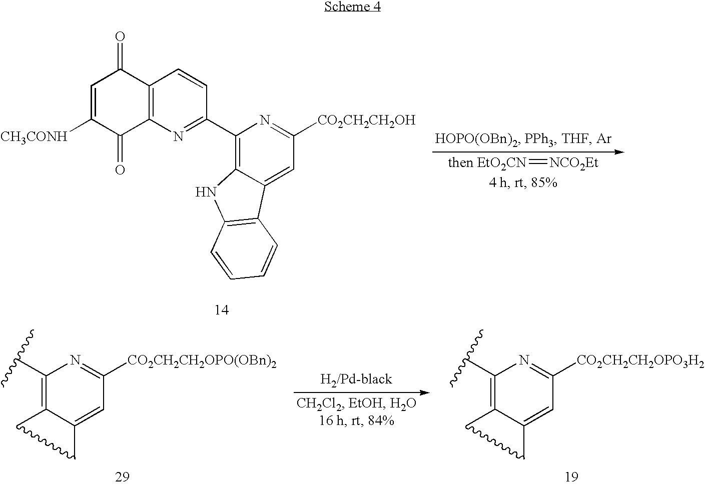
Anti-HIV Reverse Transcriptase and In Vitro and In Vivo Toxicity Studies

The lavendamycin analogs 11, 12, 16, and 18-21 were tested for their ability to inhibit HIV reverse transcriptase using a modified assay of Okada et al (e.g., see: Okada, H.; Mukai, H.; Inouye, Y; Nakamura, S. “Biological Properties of Streptonigrin Derivatives II. Inhibition of Reverse Transcriptase Activity,” J. Antibiot., 1986, 39, 306-308). Briefly, the analogues were initially dissolved in either DMSO or HEPES buffer (pH 7.2) and then diluted to the appropriate concentration before addition to the assay mixtures. Varying concentrations of either AZT-TP or the analogues or both were added to the wells of a 24-microtiter plate containing 1 unit/mL HIV-RT, ³HTTP, the template primer [poly(rA)-oligo (dT)₁₂₋₁₈], and appropriate salts and buffers. Several control wells without any inhibitors were also included on each microtiter plate. The reactions were allowed to proceed for 1 h at 37° C. The amount of radioactivity incorporated under each experimental condition was compared to that found in the control wells and the amount of inhibition of the HIV-RT was calculated for each analogue and, for certain experiments, the inhibition found with each combination of analogue and AZT-TP. A more detailed description of reaction conditions is provided below in the Examples. The concentration of drug which inhibited 50% of the enzyme activity (IC₅₀) was determined by using the computer software program Calcusyn, which plots the dose-effect curves from the inhibition data and calculates the IC₅₀ for each drug.

The results of this screen clearly show that several of these analogs have significant anti-HIV RT activity at levels considerably below the level which is cytotoxic as measured either with murine lymphocytes or human lymphocytic cell lines H-9 and CEM (Table 3).

TABLE 3
Inhibitory Activity of Lavendamycin Analogues
on HIV-RT and Cellular Cytotoxicity

| | | Mean CC₅₀ (μM)^{b} | Mean CC₅₀ (μM)^{c} |
|---|---|---|---|
| | Mean IC₅₀ | (Normal Mouse | (Human Cell |
| | (μM)^{a} ± SEM | Spleen) ± SEM | Lines) ± SEM |
| Compound | (no. of expts) | (no. of expts) | (no. of expts) |
| 11 | 15.1 ± 5.9 (3) | 62 ± 5 (4) | 31 ± 2 (4) |
| 12 | 5.8 ± 1.5 (4) | 81 ± 17 (4) | 50 ± 15 (4) |
| 16 | 12.7 ± 5.2 (6) | 50 ± 7 (4) | 95 ± 45 (4) |
| 18 | 13.3 ± 4.4 (3) | 47 ± 8.5 (4) | 81 ± 35 (4) |
| 19 | 3.0 ± 0.5 (3) | 29 ± 5 (4) | 27 ± 3 (4) |
| 20 | 7.1 ± 2.7 (5) | 48 ± 10 (4) | 47 ± 14 (4) |
| 21 | 20.5 ± 5 (3) | 15 ± 2 (4) | 17 ± 3 (4) |

^{a}Mean concentration of analogue at which 50% inhibition of HIV RT activity toward 1 unit of enzyme occurred
^{b}Mean concentration of analogue at which normal mouse spleen stimulated with concanavalin A was inhibited by 50%
^{c}Mean concentration of analogue at which human lymphocytic cell lines H-9 and CEM were inhibited by 50%

Analogs 12, 19, and 20 were the most effective HIV-RT inhibitors with IC₅₀s below 8 μM. One of the two more soluble analogs, namely the dihydrogen phosphate 19, had the greatest activity with 15 μM inhibiting the HIV-RT>92% with an IC₅₀ of 3 μM. While neither desiring to be bound by any particular theory, nor intending to limit in any measure the scope of the appended claims or their equivalents, it is presently believed that the increased water solubility of this compound due to the dihydrogen phosphate side arm allows for better interaction with the active site of the enzyme. With the exception of compound 21, the concentrations at which 50% cytotoxicity (CC₅₀s) in tissue culture were observed for the analogues were considerably higher than the IC₅₀s toward both normal mouse splenocytes and human lymphocytic cell lines. Furthermore, the levels of cytotoxicity for both human and murine cells were comparable in most cases which allows for further testing in both murine and human systems.

At 10 and 20 μM concentrations, all the analogs had a reversible inhibitory effect on normal Con A-stimulated murine spleen cells. That is, when the drugs were removed from the cultures after 12 hours, the growth resumed at the same level as unexposed control cells. This suggests that at the levels tested, the drugs are cytostatic rather than cytocidal. As the human cells studied are lines permissive for HIV replication and commonly used to examine the effectiveness of potential anti-HIV therapeutic agents, these compounds may be readily evaluated for anti-HIV activity in culture (e.g., see: Coates, A. V.; Cammack, N.; Jenkinson, H. J.; Jowett, A. J.; Jowett, M. L; Pearson, B. A.; Penn, C. R.; Rouse, P. L.; Viner, K. C.; Cameron, J. M. “(−)-2′-Deoxy-3′-Thiacytidine Is A Potent, Highly Selective Inhibitor of Human Immunodeficiency Virus Type 1 and Type 2 Replication In Vitro,” Antimicrob. Agents and Chemother., 1992, 36, 733-739; and Bilello, J.; Bauer, G.; Dudley, M. “Effect of 2′,3′-didhydro-3′-deoythymidine in an In Vitro Hollow Fiber Pharmacodynamic Model System Correlates with Results of Dose-Ranging Clinical Studies,” Antimicrob. Agents and Chemother, 1994, 38, 13 86-13 91).

Furthermore, we found that BALB/c mice both survived and tolerated the highly active anti-HIV RT analog 19 with little weight loss when given either 50 mg/kg as one ip injection or 75 mg/kg given ip in two injections within a twenty-four hour period. In other studies, we have found that several of the lavendamycin analogs have remarkably low animal toxicity. For example, the National Cancer Institute found that in mice the maximum tolerated dose of 21 was 400 mg/kg and 15 was tolerated in SCID mice at 300 mg/kg/day for 10 days with no animal death or loss of weight. Interestingly, lavendamycin and its analogs appear to be much less toxic in experimental animals than the related streptonigrin and its analogs (e.g., see: Balitz, D. M.; Bush, J. A.; Bradner, W. T.; Doyle, T. W.; O'Herron, F. A.; Nettleton, D. E. “Isolation of Lavendamycin, a New Antibiotic from Streptomyces lavendulae,” J. Antibiot., 1982, 35, 259-265; Reilly, H. C.; Sugiura, K. “An Antitumor Spectrum of Streptonigrin,” Antibiot. Chemother., 1961, 11, 174-177; and Olesen, J. J.; Calderella, L. A.; Mjos, K. J.; Reith, A. R.; Thie, R. S.; Toplin, I. “Effects of Streptonigrin on Experimental Tumors,” Antibiot. Chemother., 1961, 11, 158-164).

Although the maximum tolerated dose of the naturally occurring parent lavendamycin in mice has been reported to be 12.8 mg/kg, streptonigrin, which also has anti-HIV RT activity, is lethal to mice at doses as low as 0.4 mg/kg. The substantially lower level of animal toxicities of lavendamycins as compared to streptonigrin, another quinolinedione, may be due to the presence of the β-carboline moiety in the lavendamycins. Other quinonoid agents such as mitomycin C and adriamycin are toxic at levels far below those we have observed with the lavendamycins. In one reported tumor treatment trial in mice, the maximum tolerated dose of mitomycin C was less than 2.0 mg/kg when given for five consecutive days (e.g., see: Kim, J. Y.; Su, T. -L.; Chou, T. -C.; Koehler, B.; Scarborough, A.; Ouerfelli, O.; Watanabe, K. A. “Cyclopent[α] anthraquinones as DNA-intercalating Agents with Covalent Bond Formation Potential; Synthesis and Biological Activity,” J. Med. Chem., 1996, 39, 2812-2818). Likewise, adriamycin (doxorubicin) reportedly has a maximally tolerated dose below 10 mg/kg when given to mice once a week for three weeks (e.g., see: Casazza, A. M.; Savi, G.; Pratesi, G.; Di Marco, A. “Antitumor Activity in Mice of 4′-Deoxydoxorubicin in Comparison with Doxorubicin,” Eur. J. Cancer Clin. Oncol., 1983, 19,411-418).

Combined Activity of Analogs with Azidothymidine Triphosphate

As combined drug therapy is an essential feature of AIDS treatment today, it is important to assess the effect that any new drug may have on the activity of other mainstay anti-HIV treatments such as AZT. Thus, we examined in a checkerboard fashion the combined effect of lavendamycin analogs embodying features of the present invention with AZT-triphosphate (AZT-TP) over a range of concentrations for both drugs. The results of this evaluation are presented in Table 4 and represent two separate trials for each of the analogs 11, 12, 16, and 18-21.

TABLE 4
Combination Indices Determined for Lavendamycin
Analogs (μM) Performed by Checkerboard Analysis
Lavendamycin Analogues (μM)

| AZT | | 11 | | | | 12 | | | | 16 | | | |
|---|---|---|---|---|---|---|---|---|---|---|---|---|---|
| (μM) | | 0.25 | 0.5 | 0.75 | 1.5 | 0.25 | 0.5 | 0.75 | 1.5 | 0.25 | 0.5 | 0.75 | 1.5 |
| 0.007 | Trial 1 | 0.57 | 0.81 | 1.30 | 1.40 | 0.82 | 0.88 | 1.60 | 1.30 | 0.82 | 0.54 | 1.50 | 0.66 |
| | Trial 2 | 0.48 | 0.71 | 1.00 | 1.23 | 0.83 | 0.71 | 1.30 | 1.20 | 0.80 | 0.65 | 1.61 | 0.67 |
| 0.01 | Trial 1 | 0.94 | 0.84 | 1.20 | 1.40 | 0.58 | 0.76 | 0.86 | 0.81 | 0.85 | 0.87 | 1.0 | 1.0 |
| | Trial 2 | 0.89 | 0.76 | 1.08 | 1.12 | 0.58 | 0.76 | 0.99 | 0.91 | 1.18 | 0.70 | 1.20 | 1.0 |
| 0.02 | Trial 1 | 0.90 | 0.72 | 0.94 | 1.17 | 0.59 | 0.85 | 1.00 | 1.10 | 0.67 | 0.58 | 1.10 | 1.00 |
| | Trial 2 | 0.68 | 0.84 | 0.84 | 1.08 | 0.50 | 0.93 | 0.92 | 0.96 | 0.75 | 0.60 | 1.10 | 1.00 |
| AZT | | 18 | | | | 19 | | | | 20 | | | |
| (μM) | | 0.25 | 0.5 | 0.75 | 1.5 | 0.75 | 1.5 | 3 | 6 | 0.25 | 0.5 | 0.75 | 1.5 |
| 0.007 | Trial 1 | 0.39 | 0.40 | 0.66 | 1.00 | 0.47 | 0.49 | 0.49 | 0.32 | 0.65 | 0.62 | 0.68 | 0.83 |
| | Trial 2 | 0.37 | 0.42 | ND | 0.94 | 0.68 | 0.81 | 0.79 | 0.60 | 0.61 | 0.61 | 0.66 | 0.83 |
| 0.01 | Trial 1 | 0.44 | 0.51 | 0.59 | 0.90 | 0.64 | 0.69 | 0.28 | 0.28 | 0.77 | 0.60 | 0.64 | 0.70 |
| | Trial 2 | 0.45 | 0.50 | 0.57 | 0.82 | 1.04 | 0.69 | 0.56 | 0.75 | 0.77 | 0.61 | 0.63 | 0.69 |
| 0.02 | Trial 1 | 0.59 | 0.97 | 0.95 | 2.00 | ND | ND | ND | ND | 0.97 | 0.86 | 0.61 | 0.64 |
| | Trial 2 | 0.60 | 0.98 | 0.97 | 2.20 | ND | ND | ND | ND | 1.00 | 0.88 | 0.64 | 0.63 |

^{a} In two separate trials six different lavendamycin analogs were combined in differing ratios with AZT-triphosphate and added to HIV-RT enzyme assays in triplicate. The combination index (CI) of each drug combination was analyzed by the method of Chou and Talalay. CI values <1, 1, and >1 indicate synergism, additive effects, and antagonism. Values <0.7 are considered to be synergistic and are shown as bold. Values >0.71 to <0.9 are considered to be moderately or slightly synergistic and are shown in italics.

The results were analyzed by the method of Chou and Talalay based on the median-effect principle and represented by combination indices (CI) determined for each of the two drug combinations (e.g., see: Chou, T. C.; Talalay, P. “Quantitative Analysis of Dose-Effect Relationships: The Combined Effects of Multiple Drugs or Enzyme Inhibitors,” Adv. Enzyme Regul., 1984, 22, 27-65; and Chou, J.; Chou, T. C. “Dose-Effect Analysis with Microcomputers: Quantitation of ED50, LD50, Synergism, Antagonism, Low-Dose Risk, Receptor-Ligand Binding and Enzyme Kinetics,” In: A computer Software for IBM-PC and Manual, Cambridge, UK, Elsevier-Biosoft, 1987). This method involves the plotting of dose-effect curves for each agent and for each of the different combinations of the agents. The slope of the median-effect plot, which signifies the shape of the dose-effect curve, and the x intercept of the plot, which signifies the potency of each compound alone and each combination, were then used for a computerized calculation of a combination index. Concentrations of AZT-TP were used which were both slightly below and slightly above the IC₅₀ (0.012 μM) and concentrations of the lavendamycins were well below the CC₅₀ of each of the analogues.

Many combinations of the lavendamycins with AZT-TP were found to be either synergistic (CI≦0.7) or moderately of slightly synergistic (CI≧0.7≦0.9) particularly at the lower concentrations of the analogues. Additive effects were also seen for many of the combinations and some antagonism for some combinations, particularly at the higher concentrations of both drugs (e.g., 18 at 1.5 μM together with AZT-TP at 0.02 μM). The greatest amount of synergism over a wide range of concentrations was observed with compounds 18-20. Interestingly, the increased water solubility of compounds 18 and 19 may play a role in the increased synergism with AZT-TP (see Examples below for solubilization of analogues). Clearly, this entire series of lavendamycin analogs have the ability to inhibit the HIV-RT together with AZT in either an additive or synergistic manner.

In order to derive the dose reduction index of several of these compounds the method of Chou and Talalay was again employed using the classic isobologram technique. This method also involves the plotting of dose-effect curves for each agent as well as for multiply diluted fixed-ratio combinations of the agents. The inhibitory capacity of 12 and 18-20 with AZT-TP were evaluated by this method and the determination of CIs at 50% and 70% effect level as well as the dose reduction index (DRI) at 50% inhibition is shown in Table 5.

TABLE 5
Two Drug Combination of AZT and Lavendamycin
Analogues at Constant Molar Ratios

| | | CI at HIV-RT | | Dose Reduction | |
|---|---|---|---|---|---|
| Molar Ratio | | Inhibition of:^{a} | | Index^{b} (50%) | |
| AZT:analogue | | 50% | 70% | AZT-TP | analogue |
| 12 | 1:600^{c} | 0.45 ± 0.06 | 0.32 ± 0.08 | 3.0 | 8.2 |
| | 1:857^{d} | 0.39 ± 0.06 | 0.32 ± 0.09 | 3.9 | 7.3 |
| 18 | 1:600 | 1:03 ± 0.29 | 1.35 ± 0.64 | 1.5 | 2.6 |
| | 1:857 | 1:14 ± 0.49 | 1.7 ± 1.49 | 1.7 | 2.0 |
| 19 | 1:600 | <0.1 ± 0.10 | <0.1 ± 0.16 | >100 | >100 |
| | 1:857 | <0.1 ± 0.15 | 0.21 ± 0.18 | >40 | >15 |
| 20 | 1:214^{e} | 0.46 ± 0.11 | 0.56 ± 0.22 | 4.08 | 4.63 |
| | 1:600 | 0.49 ± 0.17 | 0.40 ± .09 | 2.4 | 13.7 |

^{a}Combination index at the effect level of 50% and 70% as calculated by the method of Chou and Talalay using a computer software program on an IBM PC. Additive effect (CI = 1), synergism (CI < 1), or antagonism (CI > 1).
^{b}Dose reduction index is a measure of how much (how many times less) the dose of each drug in a synergistic combination may be reduced at the 50% effect level compared with the doses of each drug alone.
^{c}At least four different combinations of the analogues and AZT-TP were tested for HIV-RT inhibition whereby the ratio of the two drugs was kept constant at 1:600 but the concentrations increased from 0.005 μM AZT-TP and 3 μM analogue to 0.04 μM AZT-TP and 24 μM analogue.
^{d}At least four different combinations of the analogues and AZT-TP were tested for HIV-RT inhibition whereby the ratio of the two drugs was kept constant at 1:857 but the concentrations increased from 0.0035 μM AZT-TP and 3 μM analogue to 0.28 μM AZT-TP and 24 μM analogue.
^{e}Four different combinations of analogues were tested whereby the ratio of the two drugs was kept constant at 1:214 but the concentrations increased from 0.0035 μM AZT-TP and 0.75 μM analogue to 0.028 μM AZT-TP and 6 μM analogue.

The dose reduction index is a measure of how much the dose of each drug in a synergistic combination may be reduced at a given effect level compared with the doses of each drug alone. The combination of AZT-TP and each of the four analogs 12, 18, 19, and 20 leads to considerable dose reduction with the highly soluble analog showing both the highest level of synergism at the 50^{th} and 70^{th} percent inhibition level and the greatest dose reduction index in this in vitro system. These compounds, particularly those with greater water solubility, may also show similar activity toward the replication of HIV in tissue culture and these or other derivatives may become useful adjuncts to our non-nucleoside anti-HIV arsenal.

As part of our long term research on the synthesis and biological activity studies of lavendamycin analogues as antitumor and anti-HIV agents, we have also discovered a series of analogues with various substituents at the C-6 position. These compounds are the first examples of C-6 substituted lavendamycins and are part of a much larger series of derivatives with a wide range of molecular reduction potentials and lipophilicity. Moreover, novel and efficient syntheses for making these analogues have also been discovered and will be further described below. Further description of these compounds and methods is provided in the article entitled “Total Synthesis of Novel 6-Substituted Lavendamycin Antitumor Agents” by Hassan Seradj et al. (Org. Lett.; 2004; 6(4) pp 473-476), the entire contents of which are incorporated herein by reference, except that in the event of any inconsistent disclosure or definition from the present application, the disclosure or definition herein shall be deemed to prevail.

A third series of lavendamycin analogues embodying features of the present invention have a general structure (C):

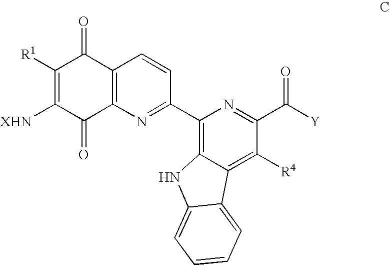

or a pharmaceutically acceptable salt thereof, wherein: X is hydrogen or R¹⁰C(O)—; R¹ is amino, alkoxy, halo, acyl or heterocyclic; Y is hydroxy, alkoxy, or amino; R⁴ is hydrogen, alkyl, aryl, cycloalkyl, alkenyl or alkynyl; and R¹⁰ is hydrogen or a substituted or unsubstituted radical selected from the group consisting of alkyl, aryl, cycloalkyl, alkenyl, alkynyl, heteroalkyl, heterocyclic, heteroalkenyl, and heteroalkynyl. In some embodiments, R¹⁰ is hydrogen, methyl, ethyl or n-propyl. In some embodiments, R¹ is —Cl, —Br, —I, —OCH₃, —OCH₂CH₃, —OCH₂CH₂CH₃, —O-n-C₄H₉, aziridinyl, pyrrolidinyl, piperidinyl or pyridinyl. In some embodiments, R¹ is 1-aziridinyl, 1-pyrrolidinyl, 1-piperidinyl or 1-pyridinyl. In some embodiments, Y is alkoxy, —NH₂, —NHR¹⁰, —NR¹⁰R¹¹ (where R¹¹ is defined as in R¹⁰, and wherein R¹¹ is the same as or different than R¹⁰). In some embodiments, Y is —NH₂, —OCH₃, —OCH₂CH₃, —OCH₂CH₂CH₃, —O-n-C₄H₉, —O-iso-amyl or —O-n-C₈H₁₇. In some embodiments, R⁴ is hydrogen or methyl.

Table 6 shows a series of 6-substituted lavendamycin analogues 30-42 embodying features of the present invention.

TABLE 6
6-Substituted Lavendamycins and Demethylavendamycins

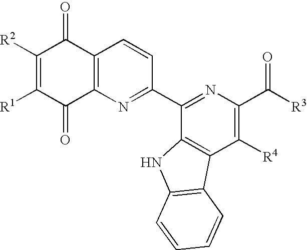

| cmpd. | R¹ | R² | R³ | R⁴ |
|---|---|---|---|---|
| 30 | NH₂ | OCH₃ | OCH₃ | H |
| 31 | NH₂ | OCH₃ | OCH₃ | CH₃ |
| 32 | NH₂ | OCH₃ | NH₂ | H |
| 33 | NH₂ | Cl | OCH₃ | CH₃ |
| 34 | NH₂ | Cl | OEt | H |
| 35 | NH₂ | Cl | 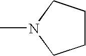 | H |
| 36 | NH₂ | Cl | Olsoamyl | H |
| 37 | NH₂ | Cl | OOctyl | H |
| 38 | NH₂ | Cl | NH₂ | H |
| 39 | AcHN | 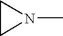 | OCH₃ | CH₃ |
| 40 | AcHN | 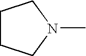 | OCH₃ | CH₃ |
| 41 | AcHN | 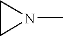 | NH₂ | H |
| 42 | AcHN | 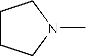 | NH₂ | H |

The compounds shown in Table 6 are the 6-substituted derivatives of our previously synthesized lavendamycins 2b, 11, 16, and 21 (e.g. see: Behforouz, M.; Gu, Z.; Cai, W.; Horn, M. A.; Ahmadian, M. J. Org. Chem. 89; Behforouz, M.; Haddad, J.; Cai, W.; Arnold, M. B.; Mohammadi, F.; Sousa, A. C.; Horn, M. A. J. Org. Chem. 1996, 61, 6552; Behforouz, M.; Haddad, J.; Cai, W.; Gu, Z. J. Org. Chem. 1998, 63, 343; Fang, Y.; Linardic, C. M.; Richardson, D. A.; Cai, W.; Behforouz, M.; Abraham, R. T. Mol. Cancer Ther. 2003, 517; and Behforouz, M.; Merriman, R. L. U.S. Pat. No. 5,525,611 issued Jun. 11, 1996). Our studies have shown compounds 2b, 11, 16, and 21 to be highly active antitumor agents.

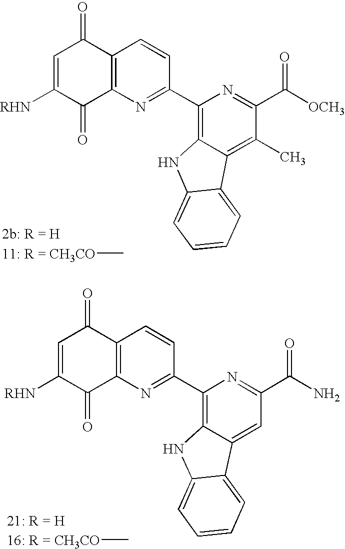

The key step in these syntheses is a Pictet-Spengler condensation of the corresponding aldehydes with the appropriate tryptophans (Scheme 5). Previously, we have reported efficient syntheses of compounds 11 (e.g., see: Behforouz, M.; Gu, Z.; Cai, W.; Horn, M. A.; Ahmadian, M. J. Org. Chem. 1993, 58, 7089; and Behforouz, M.; Haddad, J.; Cai, W.; Arnold, M. B.; Mohammadi, F.; Sousa, A. C.; Horn, M. A. J. Org. Chem. 1996, 61, 6552) and 16 (e.g., see: Behforouz, M.; Merriman, R. L. U.S. Pat. No. 5,525,611, issued Jun. 11, 1996) via this condensation reaction.

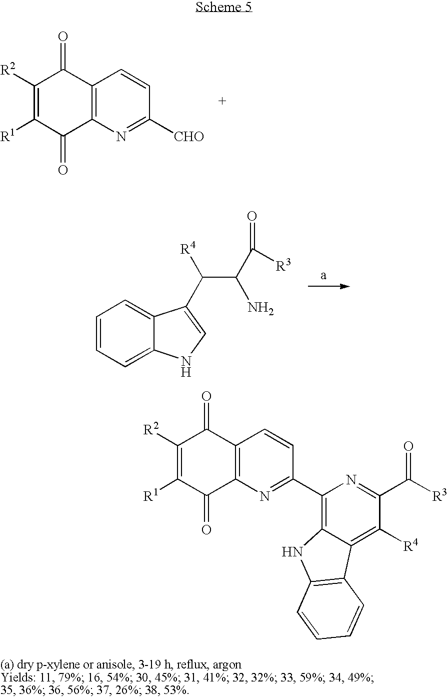

Novel carbaldehyde 49 required for the syntheses of 30, 31 and 32 was prepared according to Scheme 6.

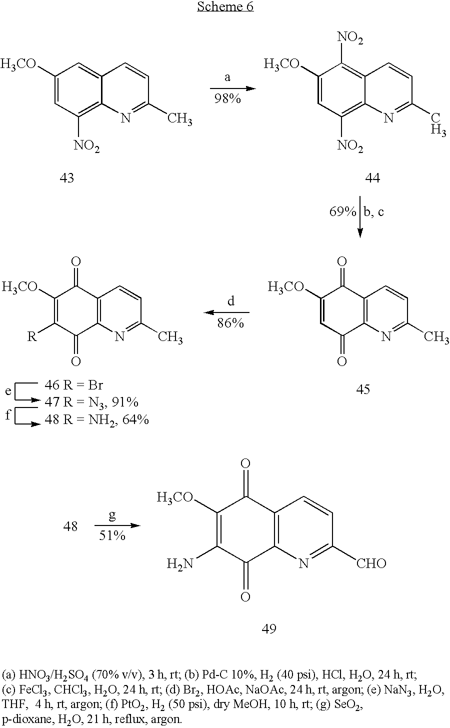

7-Amino-6-methoxy-2-methylquinoline-5,8-dione (48) was prepared (e.g., see: Liao, T. K.; Nyberg, H.; Cheng, C. C. J. Heterocyclic Chem. 1976, 13, 1063; and Boger, D. L.; Duff, S. R.; Panek, J. S.; Yasuda, M. J. Org. Chem. 1985, 50, 5782) and then oxidized to the desired aldehyde (49) via a method similar to that previously reported by us for the preparation of 3 (e.g., see: Behforouz, M.; Gu, Z.; Cai, W.; Horn, M. A.; Ahmadian, M. J. Org. Chem. 1993, 58, 7089). A controlled amount of bromine should be used in the bromination of 45. Use of more than 1.25 equivalents of bromine lowers the yield of 22 and produces undesired side products.

Carbaldehyde 3 used for the synthesis of analogs 39-42 was prepared according to Scheme 7 and the novel aldehyde 52 used for the synthesis of analogs 33-38 was obtained by the oxidation of chlorodione 51 (e.g., Behforouz, M.; Haddad, J.; Cai, W.; Gu, Z. J. Org. Chem. 1998, 63, 343) via a similar method to that used for 3.

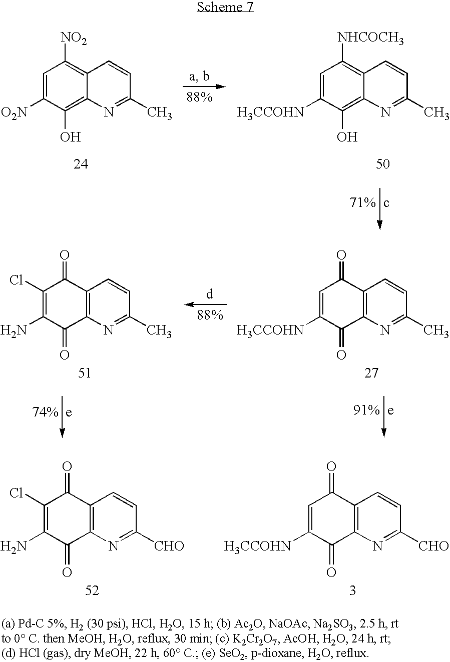

7-Acetamidoquinoline-5,8-dione 27 was converted to chloroquinone 51 in a sequence of reactions shown in Scheme 8 (Behforouz, M.; Haddad, J.; Cai, W.; Gu, Z. J. Org. Chem. 1998, 63, 343).

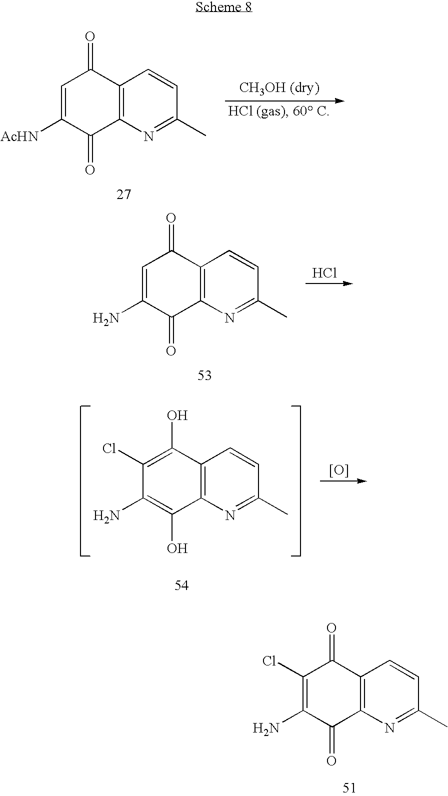

Acid-catalyzed methanolysis of 27 first gave the aminodione derivative 53. A Michael addition of HCl to aminodione 53 produced the intermediate hydroquinone 54 which then oxidized to the final 6-chloroquinone 51.

Tryptophan pyrrolidine amide (for compound 35, Scheme 5) was prepared according to the method of Tolstikov et al. (e.g., see: Tolstikov, V. V.; Holpnekozlova, N. V., Oreshkina, T. D.; Osipova, T. V.; Preobrazhenskaya, M. N.; Sztarisckai, F.; Balzarini, J.; Declercq, E. J. Antibiot. 1992, 45, 1020). β-Methyltryptophan methyl ester (for compounds 31, 33, 39, 40 and 44, Scheme 5) was prepared according to our own procedure (e.g., see: Behforouz, M.; Zarrinmayeh, H.; Ogle, M. E.; Riehle, T. J.; Bell, F. W. J. Heterocyclic Chem. 1988, 25, 1627). Tryptophan isoamyl ester (for compound 36, Scheme 5) was prepared in 82% yield by the Fischer esterification reaction in the presence of HCl gas followed by the neutralization of the resulting salt with ammonium hydroxide and EtOAc extraction. The remaining tryptophan derivatives were prepared by the neutralization of the commercially available salts.

7-Acetamido-6-alkylaminolavendamycins (39-42) were synthesized using a novel and simple method in accordance with the present invention.

A method embodying features of the present invention for synthesizing a compound having a formula: 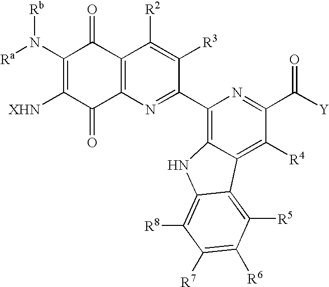
includes reacting a compound having a structure: 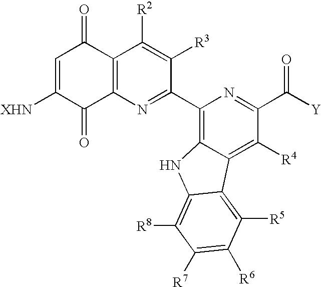
with a compound having a structure: 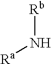
wherein:

X is hydrogen or R¹⁰C(O)—;

Y is hydroxy, alkoxy or amino;

R^{a} and R^{b} are either (a) independently hydrogen or a substituted or unsubstituted radical selected from the group consisting of alkyl, aryl, cycloalkyl, alkenyl, alkynyl, heteroalkyl, heterocyclic, heteroalkenyl, and heteroalkynyl or (b) together with the nitrogen atom to which they are attached form a three-, four-, five-, six-, seven- or eight-membered cyclic ring;

R¹ is amino, aziridinyl, pyrrolidinyl, piperidinyl or pyridinyl; R², R³, R⁴, R⁵, R⁶, R⁷, and R⁸ are each independently hydrogen or a substituted or unsubstituted radical selected from the group consisting of alkyl, aryl, cycloalkyl, alkenyl, alkynyl, heteroalkyl, heterocyclic, heteroalkenyl, heteroalkynyl, halo, nitro, cyano, alkoxy, amino, amido, thioamido, acyl, thioacyl, and imino; and

R¹⁰ is hydrogen or a substituted or unsubstituted radical selected from the group consisting of alkyl, aryl, cycloalkyl, alkenyl, alkynyl, heteroalkyl, heterocyclic, heteroalkenyl, and heteroalkynyl.

Direct addition of aziridine or pyrrolidine to compounds 11 or 16 under very mild reaction conditions resulted in the formation of almost 100% pure products (Scheme 9). No transamination occurs at the C-2′ amide and ester linkages or even at the vinylogous C-7 amide function during the course of this reaction.

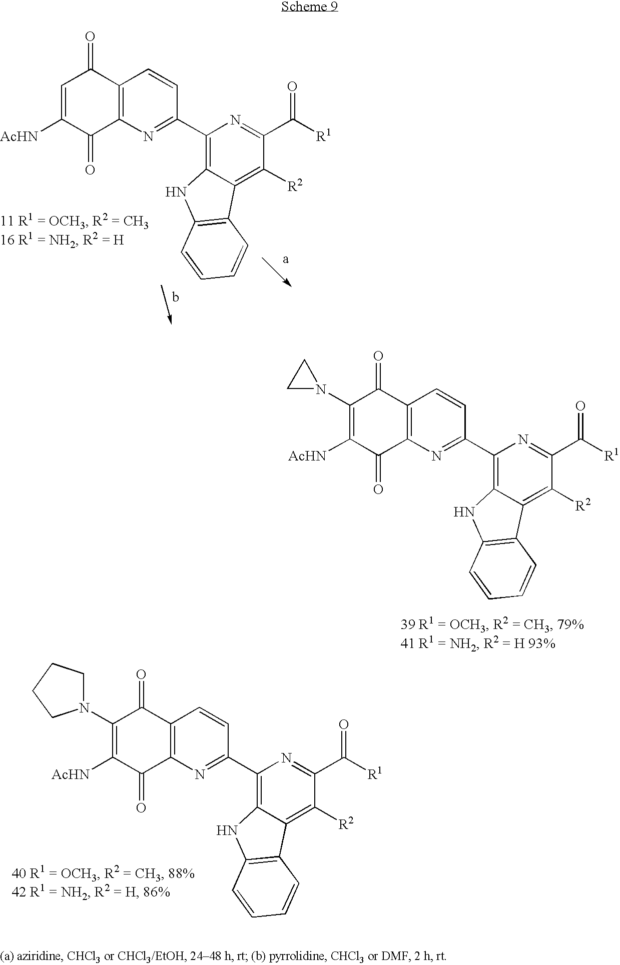

The literature lacks efficient methods for the synthesis of 6,7-diaminoquinolinediones. The use of dihalodiones (e.g., see: Choi, H. Y.; Kim, D. W.; Chi, D. Y.; Yoon, E. Y.; Kim, D. J. J. Org. Chem. 2002, 67, 5390 and references cited therein) or their methoxy derivatives (e.g., see: Yoo, K. H.; Yoon, E. Y.; Park, Y. Y.; Park, S. W.; Lee, C. -O.; Lee, W. K.; Chi, D. Y.; Kim, D. J. Bull. Korean Chem. Soc. 2001, 22, 1067) as the starting materials for the amination of this system gives a mixture of 6- and 7-amino products and not the desired diamino compound. This may be due to the fact that once the replacement of either the C-6 or the C-7 groups occurs by an amine, the resulting quinone system becomes deactivated toward the second Michael addition of another amine molecule as we have observed in compound 53.

This novel and efficient method produces the 6-aminolavendamycins as well as the 6,7-diaminodiones in excellent yields at room temperature. In addition, we have found that a variety of aliphatic and aromatic amines may be added at the C-6 positions of lavendamycins and 7-aminoquinolinediones. It appears that placement of an acyl group on the C-7 amino function enhances the reactivity at the C-6 position and facilitates the amination reaction with no necessity for the presence of halogen or alkoxy as activating groups.

While neither desiring to be bound by any particular theory, nor intending to limit in any measure the scope of the appended claims or their equivalents, it is presently believed that the mechanism for this reaction may be as presented in Scheme 10.

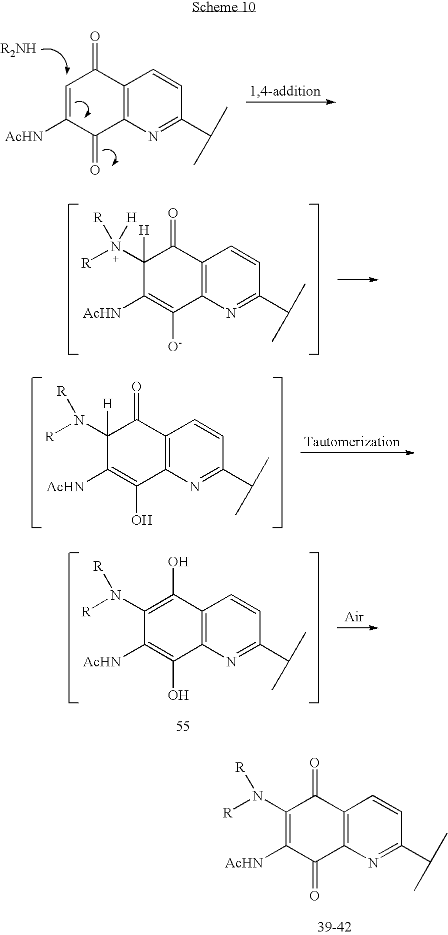

In contrast to the unreactivity of the 7-aminoquinolinedione 53 toward the amination reaction at the C-6 position, placement of an acetyl group on the amino nitrogen (e.g. 44 and 46) decreases its electron donating ability and thus activates the system toward a facile Michael addition reaction of amines. However, in the case of 1,4-addition of HCl to the aminodione 53 (Scheme 8), this transformation may be facilitated through the intermediacy of activated species such as 56 or 57.

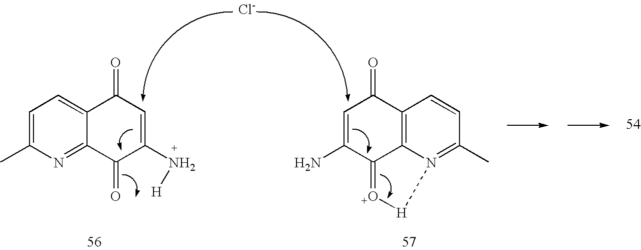

Interestingly, the spontaneous and complete reoxidation of the aminohydroquinone intermediate 55 to its corresponding quinoline-5,8-dione occurs during the reaction work-up. In contrast, the conversion of chlorohydroquinone 54 to its dione 51 results in only partial air oxidation during the reaction work-up and ferric chloride is needed to complete the reaction (e.g., see: Behforouz, M.; Haddad, J.; Cai, W.; Gu, Z. J. Org. Chem. 1998, 63, 343).

Thus, as described above, the present invention provides efficient syntheses of the first examples of 6-substituted lavendamycins, and provides a novel method for the facile introduction of an amino group at the C-6 position of a lavendamycin system.

The following representative procedures and Examples are provided solely by way of illustration, and are not intended to limit the scope of the appended claims or their equivalents. Additional procedures and Examples in accordance with the present invention are provided in U.S. patent application Ser. No. 10/233,050 filed Aug. 29, 2002, the entire contents of which are incorporated herein by reference, except that in the event of any inconsistent disclosure or definition from the present application, the disclosure or definition herein shall be deemed to prevail.

### EXAMPLES

### HIV-RT Assay

The assay procedure described below is adapted from Okada et al. (Okada, H.; Mukai, H.; Inouye, Y; Nakamura, S. “Biological Properties of Streptonigrin Derivatives II. Inhibition of Reverse Transcriptase Activity,” J. Antibiot., 1986, 39, 306-308). The lavendamycin analogs were initially dissolved in DMSO at a concentration of 1 mg/mL. Occasionally, mild sonication and heat were used to dissolve the less soluble compounds. The dissolved analogs were then serially diluted in distilled water to achieve concentrations in the final reaction mixture ranging from 0.5-15 μM. In addition to the lavendamycin analogs, the final reaction mixture contained 100 mM Tris-HCl (pH 8.0), 5 mM MgCl₂, 5 mM dithiothreitol, 60 mM NaCl, 0.2 mM [³H] thymidine triphosphate(5 μCi well), 4 μg/mL poly (rA)-oligo(dT)₁₂₋₁₈ (Amersham Pharmacia Biotech, Piscataway, N.J.) and 1 unit/mL of HIV-RT (Worthington Biochemical Corp., Freehold, N.J.) in a total volume of 300 μL. For each assay, the reaction mixture containing 8× buffer, double distilled H₂O, ³HTTP, template-primer and HIV-RT was pre-mixed in a 15-mL sterile tube in quantities required for the particular experiment. After adding the enzyme, the reaction mixture was mixed and kept on ice. The inhibitors, either AZT(Moravek Biochemicals, Brea, Calif.), analogs or both were added to appropriate wells in a 24 well microtiter plate and ddH₂O was added to each well to bring the volume of all test wells to 100 μL. Two hundred μL of the chilled reaction mixture was then added to each of the test wells, mixed, and incubated for 1 hr at 37° C. Several control wells with no inhibitors were included on each test plate. After stopping the reactions with 25 μL 0.1 M EDTA, triplicate samples of 15 μL from each test well were spotted on DE81 ion exchange filter paper squares (Whatman Paper, Maidstone, England). After drying, each filter was washed for 10 minutes three times with 5% TCA-NHPO₄, three times with 0.6M NaCl-0.06 mM Na Citrate, one time with ddH₂O and one time with 95% ethanol. After drying the individual filter papers were added to 5 mL Scintiverse E (Fisher Scientific, Chicago, Ill.) scintillation fluid and the amount of 3H-TTP uptake measured as counts per minute on a Beckman liquid scintillation counter. Mean uptake obtained with triplicate wells containing inhibitors were compared with the mean uptake found in 3 sets of triplicate control wells containing enzyme plus substrate and percent inhibition for each experimental condition was calculated. The results were then analyzed using Calcusyn, a windows software program for dose effect analysis designed by T. -C. Chou and M. P. Hayball and published by Biosoft® (UK).

### Cytotoxicity Assays

Normal murine spleen cells were washed in HBSS, cultured at 2.5×10⁶ or 5×10⁶ cells per mL in RPMI 1640 (with Hepes) complete medium with 2 mM-L-glutamine, 1 mM sodium pyruvate, 0.05 mM β-mercaptoethanol, penicillin, streptomycin, and amphotericin B with 10% fetal calf serum and Concanavalin A (ConA) at 2.5 μg/mL in a 96-well microtiter plate. A range of diluted analogs were added to triplicate culture wells and the cells were incubated at 37° C. in 5% CO₂. Wells containing cells, media and ConA were used as controls. Forty-eight hours post incubation, 20 μL of the vital dye MTT (3-[4,5-Dimethylthiazol-2-yl]-2,5-diphenyltetrazolium bromide; thiazolyl blue) at 5 mg/mL was added to each well and the cultures re-incubated for 4-6 hours. Following centrifugation, the supernatants were decanted and the stained cells were resuspended in 100 μL of 0.4% HCl acidified isopropanol. The optical densities of each well were read at 600 nm using a Cambridge Technologies series 700 microplate reader. The human lymphocytic cell lines CCRF-CEM (ATCC: HTB 176) and H-9 (ATCC: CCL 119) were subcultured from recently cultured cells grown in complete RPMI 1640 medium as described above but without the β-mercaptoethanol and ConA at 6.25×10⁵ and 2×10⁶ per mL in a 96 well culture plate. These cell numbers represented log phase and early stationary phase of the freshly cultured cells. As these cell lines rapidly reproduce without mitogenic stimulation, Con A was not used. A range of analogues or media alone was added in triplicate to the cultures and the cultures incubated, pulsed with MTT, harvested and their growth estimated as described above for the murine cultures. The CC₅₀ or the concentration of drug which inhibited cell growth as estimated by MTT formazan production to 50% of that of untreated control cells was determined by linear interpolation for each lavendamycin analog. As the results from the cytotoxicity assays of the two human cell lines were not significantly different, the data were pooled and analyzed together.

### In Vivo Toxicity

Analogue 19 was first solubilized in 10% dry DMSO, diluted in water, and given to the mice intraperitoneally in one dose or in two doses per day. Mice were weighed and observed for 3 days following treatment.

### Chemistry—General Methods

Chemical reagents and solvents were purchased from Aldrich, Sigma, and Fisher Chemical companies and used as received unless otherwise noted. Dioxane, xylene, anisole, and THF were distilled from sodium/benzophenone. DMF was dried and distilled over calcium hydride. Ammonium formate was dried in a vacuum desiccator over calcium sulfate. Analytical TLC was performed on 0.1 mm Eastman Kodak and Baker silica gel 60 F₂₅₄ plates. Baker silica gel (60, particle size 4.0 μm) was used for flash column chromatography. NMR spectra were recorded on Varian Gemini 200 and JEOL 400 spectrophotometers and calibrated by using the residual undeuterated solvent as internal standard. Chemical shifts (δ) are in ppm and coupling constants (J) are in Hz. Infrared spectra were recorded on a Perkin-Elmer 1000 series FT-IR spectrometer. Low resolution mass spectra and HRMS were recorded on Kratos MS 80, and the relative peak intensities are given in parentheses. Elemental analyses were performed by the Midwest Microlabs. Ltd. Melting points are uncorrected. Each new compound showed a single spot TLC (SiO₂ unless indicated otherwise), was pure as shown by NMR, and gave excellent elemental analyses or HRMS data.

### 7-Acetamido-2-formylquinoline-5,8-dione (3)

This aldehyde was prepared from dinitro 24 according to our previously reported method (see Scheme 3; Behforouz, M.; Haddad, J.; Cai, W.; Arnold, M. B.; Mohammadi, F.; Sousa, A. C.; Horn, M. A. “Highly Efficient and Practical Syntheses of Lavendamycin Methyl Ester and Related Novel Quinolindiones,” J. Org. Chem., 1996, 61, 6552-6555).

### 7-Butyramido-2-formylquinoline-5,8-dione (4)

In a 25-mL round-bottomed flask equipped with a magnetic bar, a condenser, and an argon filled balloon, 7-butyramido-2-methylquinoline-5,8-dione (0.516 mg, 2 mmol), selenium dioxide (0.255 g, 2.3 mmol), 12 mL dried distilled 1,4-dioxane, and 0.25 mL water were stirred and slowly heated to reflux over two hours. The reaction mixture was refluxed for 13 hours, the black selenium metal was allowed to settle and the supernatant was pipetted off and filtered. Dioxane (10 mL) was added to the solid residue, stirred and refluxed for 5 minutes. The entire mixture was filtered and the selenium was washed with dichloromethane (10 mL). To the combined filtrates was added 50 mL dichloromethane, and they were washed with 3% sodium bicarbonate solution (2×50 mL), dried (MgSO₄) and evaporated in vacuo to give 356 mg, (65%) of a pale yellow solid. The product was recrystallized from ethyl acetate: mp 208-210° C.; R_{f}=0.39 (1/1 EtOAc/CH₂Cl₂); ¹H NMR (CDCl₃): δ 1.02 (t, 3H, J=7.4 Hz), 1.70-1.89 (m, 2H), 2.52 (t, 2H, J=7.4), 8.06 (s, 1H), 8.31 (d, 1H, J=8.0 Hz), 8.39 (br s, 1H), 8.61 (d, 1H, J=8.0 Hz), 10.28 (s, 1H); EIMS, m/z, 272 (54), 202 (36), 175 (9); Analysis for C₁₄H₁₂N₂O₄ calculated C, 61.76; H, 4.44; N, 10.29; found C, 61.31; H, 4.36; N, 9.94.

### 7-Butyramido-2-methylquinoline-5,8-dione (28)

In a 500-mL round-bottomed flask equipped with a magnetic bar, 5,7-dibutyramido-8-butyroxy-2-methylquinoline (26, 3.29 g, 8.25 mmol) was suspended in 122 mL of glacial acetic acid. A solution of potassium dichromate (8.8 g, 30 mmol) in 115 mL of water was added and stirred at room temperature. Dichloromethane (70 mL) was added to promote solution, and the resulting two-phase mixture was stirred overnight. The organic layer was separated, and the aqueous portion was extracted with dichloromethane (12×50 mL). The combined extracts were washed with 200 mL of 3% sodium bicarbonate solution, dried (MgSO₄), evaporated, and dried under vacuum to give an orange yellow solid (1.65 g, 77%). The product was recrystallized from ethyl acetate: mp 188-189° C.; ¹H NMR (CDCl₃): δ 1.00 (t, 3H, J=7.4), 1.69-1.82 (m, 2H), 2.48 (t, 2H, J=7.4), 2.74 (s, 3H), 7.53 (d, 8.0), 7.90 (s, 1H), 8.28 (d, 1H, J=8.0), 8.36 (br s, 1H); EIMS, m/z, 258 (81), 215 (7), 188 (100), 161 (66); Analysis for C₁₄H₁₄N₂O₃ calculated C, 65.11; H, 5.46; N, 10.85; found C, 65.22; H, 5.51; N, 10.90.

### 5,7-Dibutyramido-8-butyroxy-2-methylquinoline (26)

In a 500-mL heavy-walled hydrogenation bottle, 5.00 g (0.2 mmol) of finely powdered 8-hydroxy-2-methyl-5,7-dinitroquinoline (Behforouz, M.; Gu, Z.; Cai, W.; Horn, M. A., Ahmadian, M. “A Highly Concise Synthesis of Lavendamycin Methyl Ester,” J. Org. Chem., 1993, 58, 7089-7091) and 5% Pd/C (1.5 g) were suspended in 100 mL of water and 12 mL of concentrated hydrochloric acid. In a Parr Hydrogenator, this mixture was shaken under 30 psi of hydrogen for 15 hours. The catalyst was removed, and the dark red solution containing the dihydrochloride salt of 5,7-diamino-8-hydroxy-2-methykquinoline was placed in a 250-mL round-bottomed flask equipped with a magnetic bar. To the stirred solution were added, as quickly as possible, sodium sulfite (12 g), sodium acetate (16 g), and butyric anhydride (65 mL). The thick whitish solid that continued to form over a three-hour period was filtered, washed with water, and dried under vacuum (7.4 g, 93%). Attempts to recrystallize 26 from methanol-water caused it to hydrolyze to the corresponding 5,7-dibutyramido-8-hydroxyquinoline-5,8-dione. However, NMR sowed, the crude 26 to be relatively pure and it was used as such for the preparation of 28. Analytical data for 26: mp 195-200° C.; R_{f}=0.26 (0.1/5 MeOH/EtOAc); ¹H NMR (DMSO-d₆) δ 0.92 (t, 3H, J=8.0), 0.96 (t, 3H, J=8.0), 1.08 (t, 3H, J=8.0), 1.52-1.72 (m, 4H), 2.58 (s, 3H), 2.70 (t, 2H, J=8.0), 7.37 (d, 1H, J=8.8), 8.21 (d, 1H, J=8.8), 8.24 (s, 1H), 9.65 (br s, 1H), 9.94 (br s, 1H).

### 5,7-Dibutyramido-8-hydroxy-2-methylquinoline

This compound was obtained by dissolving 26 in a minimum amount of hot Methanol-water (1/1) (Behforouz, M.; Haddad, J.; Cai, W.; Arnold, M. B.; Mohammadi, F.; Sousa, A. C.; Horn, M. A. “Highly Efficient and Practical Syntheses of Lavendamycin Methyl Ester and Related Novel Quinolinediones,” J. Org. Chem. 1996, 61, 6552). Pale white crystals were filtered and dried: mp 200-208° C. (dec.); R_{f}=0.32 (0.1/5 MeOH/EtOAc); ¹H NMR (DMSO-d₆) δ 0.93 (t, 3H, J=7.0), 0.96 (t, 3H, J=7.0), 2.3-2.55 (m, 4H), 2.7 (s, 3H), 7.3 (d, 1H, J=8.8), 8.03 (3, 1H), 7.37 (d, 1H, J=8.8), 9.45 (br s, 1H), 9.63 (br s, 1H); EIMS, m/z, 329 (95), 286 (20), 259 (98), 241 (17), 216 (6), 188 (100); HRMS, m/z calculated for C₁₈H₂₃N₃O₃ 329.173942 found 329.173043; Analysis calculated C, 65.63; H, 7.04; N, 12.76; found C, 65.80; H, 7.05; N, 12.75.

### Tryptophan β-Hydroxyethyl Ester (8)

In a 250-mL two-necked, round-bottomed flask was dissolved compound 23 (2.088 g, 5.4 mmol) in 100 mL dry DMF under an argon balloon. Dry ammonium formate (1.124 g, 17.82 mmol) and 964 mg of 10% Pd/C were added, and the mixture was stirred for 4.5 hours at room temperature. The reaction mixture was filtered through a layer of Celite, and the filter cake was washed with 3×8 mL of EtOAc. The light orange solution was evaporated in vacuo and dried under a vacuum pump at 50-60° C. for two or more days until no trace of DMF was observed in the product NMR. The product was a gel and weighed 1.164 g (87%); R_{f}=0.25 (0.25/5 MeOH/CH₂Cl₂); ¹H NMR (DMSO-d₆) δ 2.95 (dd, 1H, J=14.1, 6.2), 3.04 (dd, 1H, J=14.1, 6.0), 3.60-3.70 (m, 2H), 3.63 (dd, 1H, J=6.0, 6.2), 3.99-4.10 (m, 2H), 6.97 (dd, 1H, J=8.1, 7.0), 7.05 (dd, 1H, J=8.1, 7.0), 7.14 (s, 1H), 7.33 (d, 1H, J=8.1), 7.51 (d, 1H, J=8.1), 7.51 (d, 1H, J=8.1), 7.95 (s, 1H), 10.86 (br s, 1H); HRMS (EI) calculated for C₁₃H₁₆N₂O₃ 248.1161, found 248.1158.

### N-(Benzyloxycarbonyl)tryptophan β-Hydroxyethyl Ester (23)

To partially dissolved CBZ-tryptophan (22, 1.02 g, 3 mmol) in 18 mL of n-butyl ether in a 50-mL three-necked, round-bottomed flask under argon were added 2-chloroethanol (483 mg, 6 mmol) and triethylamine (0.4 mL, 291 mg, 2.9 mmol) via a syringe. The reaction mixture was refluxed overnight for 5 hours. The two-layered mixture was evaporated in vacuo, and the residue was dissolved in 12 mL of EtOAc and placed in the refrigerator overnight. The white solid material was filtered and rinsed with EtOAc, and the filtrate was washed with 5 mL of a 5% solution of sodium bicarbonate followed by water until the pH of the aqueous layer reached 7 (5×3 mL). The organic solution was dried (Mg SO₄), filtered, and evaporated in vacuo and then dried on a pump at 40-50° C. to give a dark orange gel (880.4 mg, 78%); R_{f}=0.44 (1/5 EtOH/EtOAc, Al₂O₃); ¹H NMR (CDCl₃) δ 3.20-3.40 (m, 2H), 3.55-3.70 (m, 2H), 4.00-4.25 (m, 2H), 4.60-4.80 (m, 1H), 5.00-5.15 (m, 2H), 5.25-5.45 (m, 2H), 7.02 (s, 1H), 7.05-7.15 (m, 1H), 7.17-7.25 (m, 1H), 7.25-7.4 (m, 5H), 7.50-7.60 (m, 1H), 8.09 (br s, 1H). HRMS (EI) calculated for C₂₁H₂₂N₂O₅ 382.1528, found 382.1523.

### 7-N-Acetyldemethyllavendamycin β-Hydroxyethyl Ester (14)

In a 500-mL three-necked, round-bottomed flask equipped with a Dean-Stark trap, a condenser, a dropping funnel, and a magnetic bar, 7-acetamido-2-formylquinoline-5,8-dione (3, 146.4 mg, 0.6 mmol) in 250 mL dry anisole was stirred and heated under argon to 80° C. over 30 min. Nearly all of aldehyde 3 was solubilized. To this mixture was dropwise added a solution of tryptophan β-hydroxyethyl ester (8, 198.64 mg, 0.8 mmol) in 12 mL of dry DMF over 40 minutes at 95° C., and the temperature was slowly raised to 160° C. over 4.5 hours and then heated at this temperature for an additional one hour. The reaction mixture was allowed to cool for 10 minutes and then filtered to remove a small amount of colloidal material. The filtrate was evaporated in vacuo to dryness, and then 15 mL acetone was added and allowed to stand at room-temperature overnight. The solid material was filtered and washed with acetone followed by ether to give 177 mg (94%) of yellow orange solid 14: mp 270.5-271 (dec.); R_{f}=0.69 (0.2/5 MeOH/CH₂Cl₂); ¹H NMR (DMSO-d₆): δ 2.32 (s, 3H), 3.80-3.90 (m, 2H), 4.40-4.50 (m, 2H), 7.45 (t, 1H, J=7.3), 7.72 (d, 1H, J=7.3), 7.70-7.75 (m, 1H), 7.83 (s, 1H), 8.53 (d, 1H, J=8.0), 8.61 (d, 1H, J=8.4), 8.98 (d, 1H, J=8.4), 10.31 (s, 1H), 12.01 (br s, 1H); HRMS (EI) calculated for C₂₅H₁₈N₄O₆ 470.1226 found 470.12406.

### Dibenzyl Phosphate (29)

To a stirred mixture of 14 (135.7 mg, 0.289 mmol), dibenzyl hydrogen phosphate (241.2 mg, 0.87 mmol) and triphenyphosphine (228.2 mg, 0.87 mmol) in 8 mL dry THF, under Ar, was dropwise added a solution of diethyl azidodicarboxylate (151.52 mg, 0.87 mmol) in 2 mL dry THF via a syringe over a course of 12 minutes. The mixture was stirred for 4 hours at room temperature, and the resulting solid was filtered off. The filtrate was evaporated to dryness, and the solid was washed with ethyl ether to give 179 mg (85%) of orange solid 29. Silica gel chromatography (1% MeOH in CH₂Cl₂) gave 142 mg of pure 29: mp 183.5-184° C.; R_{f}=0.46 (EtOAc); ¹H NMR (CDCl₃) δ 2.36 (s, 3H), 4.45 (br s, 2H), 4.64 (br s, 2H), 5.07 (s, 2H), 5.12 (s, 2H), 7.20-7.34 (m, 10H), 7.40 (dd, 1H, J=8.0, 7.3), 7.68 (dd, 1H, J=8.0, 7.3), 7.76 (d, 1H, J=8.0), 8.01 (s, 1H), 8.20 (d, 1H, J=8.0), 8.45 (s, 1H), 8.48 (d, 1H, J=8.3), 8.99 (s, 1H), 9.18 (d, 1H, J=8.3), 11.85 (br s, 1H); ³¹p NMR (CDCl₃): δ 1.69; HRMS (EI) calculated for C₃₉H₃₄N₄O₉P (M+3) 733.2065 found 733.2065.

### Dihydrogen Phosphate (19)

To a solution of 29 (38.8 mg, 0.053 mmol) in 7.4 mL of a mixture of CH₂Cl₂/EtOH/H₂O (5/2/0.4) was added 14.3 mg of palladium black, and the mixture was stirred under a balloon full of hydrogen for 16 hours. The balloon was removed, the mixture was filtered, the filter cake was thoroughly washed with 22 mL of a 1/1 mixture of EtOH/CH₂Cl₂, and then the filtrate was stirred in the presence of air for 2 hours. The resulting filtrate was evaporated in vacuo to dryness to yield 19.6 mg (84%) of pure orange brown 19: mp 179° C. (dec.); Water solubility (0.05 mg/1 mL HEPES buffer solution); R_{f}=0.54 (Eastman Kodak SiO₂, 8/3/0.5 MeOH/H₂O/EtOAc); ¹H NMR (DMSO-d₆): δ 2.32 (s, 3H), 4.27 (br s, 2H), 4.57 (br s, 2H), 7.41 (dd, 1H, J=8.0, 7.6), 7.64 (d, 1H, J=8.0), 7.71 (dd, 1H, J=8.0, 7.6), 7.79 (s, 1H), 8.47 (d, 1H, J=7.7), 8.53 (1H, d, J=8.0), 8.91 (d, 1H, J=8.0), 9.08 (s, 1H), 10.26 (s, 1H), 11.89 (br s, 1H); ³¹P NMR (CDCl₃): δ-0.02. HRMS (FAB) calculated for C₂₅H₁₉N₄O₉P 550.0089, found 550.0912.

### 7-N-n-Butyryidemethyllavendamycin n-Butyl Ester (12)

Tryptophan n-butyl ester (6, 78.1 mg, 0.3 mmol) was dissolved in 180 mL of dry anisole. To this solution under an argon balloon was added 7-n-butyramido-2-formylquinoline-5,8-dione (81.6 mg, 0.3 mmol), and the mixture was stirred and heated to reflux over a course of three hours. The reflux was continued for another 3.5 hours, and the solution was evaporated in vacuo to dryness. The residue was dissolved in acetone and allowed to stir at room temperature in the presence of air for 24 hours. The yellow-orange product was filtered and dried under vacuum (82.3 mg, 54%): mp 220.8-221° C. (dec.); R_{f}=0.54 (0.5/5 EtOAc/CH₂Cl₂); ¹H NMR (CDCl₃) δ 0.82 (t, 3H, J=7.3), 0.95 (t, 3H, J=7.5), 1.42-1.6 (m, 4H), 1.75-1.86 (m, 4H), 4.56 (t, 2H, J=6.6), 7.15-7.35 (m, 2H), 7.56 (d, 1H, J=8.1), 7.85 (d, 1H, J=8.1), 7.89 (br s, 1H), 8.14 (d, 1H, J=8.1), 8.22 (s, 1H), 9.15 (d, 1H, J=8.1), 9.19 (s, 1H), 11.96 (br s, 1H); EIMS, m/z, 510 (M⁺, 69), 446 (12), 417 (11.5), 410 (100), 394 (36), 340 (39), 217 (13); HRMS calculated for C₂₉H₂₆N₄O₅ 510.1903 found 510.1886.

### 7-N-Acetylaminodemethyllavendamycim Benzyl Ester (13)

In a 500-mL three-necked, round-bottomed flask under argon was dissolved 7-acetamido-2-formylquinoline-5,8-dione (3, 146.4 mg, 0.6 mmol) in 300 mL of dry anisole and heated to 100° C. To this mixture was dropwise added tryptophan benzyl ester (7, 176.4 mg, 0.6 mmol) over 30 minutes. The yellow lemon solution was gradually heated to 152° C. over two hours and kept at this temperature for 18 hours. In order to convert any generated hydroquinone intermediate to the desired product, a flow of oxygen gas was passed through the lemon yellow hot solution for 20 minutes. The resulting golden yellow mixture was filtered hot to remove some impurities, evaporated in vacuo, and then left overnight in a vessel open to the air. The small amount of the brownish solid was removed, and the solution was concentrated to near dryness. Acetone (10 mL) was added, and the resulting solid material was filtered and washed with a small portion of acetone and then petroleum ether to give a pure golden crystalline solid (222 mg, 72%): mp 174.6-175° C.; R_{f}=0.68 (0.1/5 MeOH/CH₂Cl₂); ¹H NMR (CDCl₃) δ 2.36 (s, 3H), 5.55 (s, 2H), 7.35-7.48 (m, 5H), 7.58-7.62 (m, 2H), 7.64-7.7 (m, 1H), 7.72-7.78 (m, 1H), 8.00 (s, 1H), 8.23 (d, 1H, J=8.0), 8.43 (br s, 1H), 8.56 (d, 1H, J=8.4), 8.98 (s, 1H), 9.21 (d, 1H, J=8.4), 11.83 (br s, 1H); HRMS (EI) calculated for C₃₀H₂₀N₄O₅ 516.14347 found 516.14340.

### 7-N-Acetyldemethyllavendamycin (18)

To a stirred mixture of benzyl ester 13 (125.8 mg, 0.24 mmol) in dichloromethane(186 mL), methanol (14 mL), and water (0.01 mL) was added 42 mg of palladium black, and the mixture was hydrogenated under a hydrogen filled balloon for 20 hours. Palladium was removed, and the solution was stirred under oxygen-filled balloon for 96 hours and then evaporated in vacuo. The solid was washed with acetone followed by ether and then dried under vacuum to give 94.2 mg (92%) of brown orange 13: mp>270° C.; Water solubility (0.05 mg/1 L HEPES buffer solution); R_{f}=0.34 (1/2 MeOH/CH₃CN); ¹H NMR (DMSO-d₆) δ 2.32 (s, 3H), 7.43 (t, 1H, J=7.7), 7.66-7.88 (m, 2H), 7.82 (s, 1H), 8.54 (d, 1H, J=7.7), 8.58 (d, 1H, J=8.4), 9.18 (d, 1H, J=8.4), 10.28 (s, 1H), 11.99 (br s, 1H); HRMS (EI) calculated for C₂₃H₁₄N₄O₅ 426.0964 found 426.0959.

### 7-N-Acetyldemethyllavendamycin n-Octyl Ester (15)

This compound was prepared according to a method similar to that of 17. The final product was obtained by the evaporation of the reaction mixture in vacuo, and the brown solid was washed with a mixture of ethyl acetate-hexane to give the orange solid 15 in 48%: mp 210.5-211° C.; R_{f}=0.41 (0.06/5 MeOH/CH₂Cl₂); ¹H NMR (DMSO-d₆) δ 0.86 (t, 3H, J=7.3), 1.25-1.6 (m, 10H), 1.83 (m, 2H), 2.32 (s, 3H), 4.41 (t, 2H, J=6.6), 7.44 (dd, 1H, J 8.0, 7.3), 7.65-7.8 (m, 2H), 7.82 (s, 1H), 8.54 (d, 1H, J=8.0), 8.57 (d, 1H, J=8.2), 8.93 (d, 1H, J=8.2), 9.09 (s, 1H), 10.29 (s, 1H), 11.96 (br s, 1H); EIMS, m/z, 538 (M⁺, 55), 382 (100), 366 (5), 340 (16); HRMS (EI) calculated for C₃₁H₃₀N₄O₅ 538.2216 found 538.2220.

### Demethyllavendamycin n-Octyl Ester (20)

7-N-Acetyldemethyllavendamycin n-octyl ester (15, 76.3 mg, 0.142 mmol) was added to 7 mL of a 70% solution of sulfuric acid and under an argon balloon was stirred and heated at 60° C. for 45 minutes. The reaction mixture was carefully basified with a saturated solution of sodium carbonate to pH=8 and then extracted with chloroform (4×45 mL). The combined extracts were washed with water (2×20 mL), dried (MgSO₄), and concentrated to a small volume. The solution was cooled in the refrigerator and the red solid was filtered off. Silica gel flash chromatography using chloroform and then methanol-chloroform (1.6 mL/100 mL) as the solvent system gave the pure orange red product 20 (45 mg, 64%): mp 175-176° C. (dec.); R_{f}=0.12 (0.06/5 MeOH/CH₂Cl₂); ¹H NMR (CDCl₃) δ 0.96 (t, 3H, J=7.3), 1.25-1.40 (m, 10H), 1.93 (m, 2H), 4.59 (t, 2H, J=6.9), 7.10-7.30 (m, 1H), 7.35-7.43 (m, 1H), 7.51 (d, 1H, J=8.0), 7.74 (br s, 1H), 7.82 (d, 1H, J=8.0), 8.11 (d, 1H, J=8.0), 8.15 (s, 1H), 9.10 (d, 1H, J=8.0), 9.16 (s, 1H), 11.84 (br s, 1H); HRMS (EI) calculated for C₂₉H₂₈N₄O₄ 496.2110 found 496.2099.

### 7-N-Acetyldemethyllavendamycin Amide (16)

This compound was prepared according to a method similar to the procedure used for the synthesis of 17. The final product was obtained as a lemon yellow solid in 62% yield by cooling the reaction mixture in the refrigerator, filtration of the solid, and washing with hexane: mp>280° C. (dec.); R_{f}=0.65 (0.2/5 MeOH/CH₂Cl₂); ¹H NMR (DMSO-d₆) δ 2.32 (s, 3H), 7.41 (dd, 1H, J=7.5, 7.0), 7.6-7.75 (m, 3H), 7.82 (s, 1H), 8.48 (dd, 1H, J=8.4), 8.52 (d, 1H, J=8.0), 8.61 (br s, 1H), 9.07 (s, 1H), 10.29 (s, 1H), 11.95 (br s, 1H); HRMS (EI) calculated for C₂₃H₁₅N₅O₄ 425.1124 found 425.1115.

### 7-N-n-Butyramidodemethyllavendamycin Amide (17)

To a stirred solution of tryptophan amide (10, 243.6 mg, 1.2 mmol) in 480 mL of dry anisole under an argon balloon was added 7-N-butyramido-2-formylquinoline-5,8-dione (4, 326.4 mg, 1.2 mmol), and the mixture was heated to reflux over a three-hour period. The reaction mixture was refluxed for 13 hours and then allowed to stand at room temperature overnight. The yellow solid was filtered off and washed with a small volume of acetone followed by ether to give 343.3 mg (63%) of pure 17: mp 244° C. (dec.); R_{f}=0.58 (0.2/5 MeOH/CH₂Cl₂); ¹H NMR (DMSO-d₆) δ 0.95 (t, 3H, J=7.3), 1.64 (quintet, 2H, J=7.3), 2.64 (t, 2H, J=7.3), 7.41 (dd, 1H, J=8.2, 7.9), 7.70-7.76 (m, 2H), 7.83 (s, 1H), 8.47 (d, 1H, 8.2), 8.51 (d, 1H, J=7.9), 9.05 (s, 1H), 9.45 (d, 1H, 8.2), 10.18 (s, 1H), 11.92 (br s, 1H); HRMS (EI) calculated for C₂₅H₂₀N₅O₄ (M+1) 454.15153 found 454.15129.

### Demethyllavendamycin Amide (21)

7-N-n-Butyryldemethyllavendamycin amide (17, 147.8 mg, 0.32 mmol) was placed in a 50-mL two-necked, round-bottomed flask under an argon balloon. A 70% solution of sulfuric acid (7.5 mL) was dropwise added and the homogeneous mixture was stirred and heated at 60° C. in an oil bath for 6 hours. The dark red solution was cooled to 0° C. and then added to 75 mL of ice-water. The mixture was carefully basified with a saturated solution of sodium carbonate to about pH=8. The solution was evaporated in vacuo to dryness and then water (75 mL) was added and stirred. The orange red crystals were washed with water and dried under vacuum (115.4 mg, 96.5%): mp 154.2° C. (dec.); R_{f}=0.45 (0.2/5 MeOH/CH₂Cl₂); ¹H NMR (DMSO-d₆) δ 5.96 (s, 1H), 7.41 (t, 1H, J=7.3), 7.60-7.74 (m, 2H), 7.79 (d, 1H, J=8.0), 8.46 (d, 1H, J=8.0), 8.52 (d, 1H, J=7.3), 8.60 (s, 1H), 9.06 (s, 1H), 9.44 (d, 1H, J=8.0), 12.00 (br s, 1H); HRMS (EI) calculated for C₂₁H₁₃N₅O₃ 383.1018 found 383.1026.

### General Synthetic Procedures for Preparing 6-Substituted Lavendamycin Analogues

1,4-Dioxane, xylene, and anisole were dried and distilled over sodium. Ethanol and chloroform were dried and distilled over CaH₂. DMF was dried over molecular sieve. All other solvents were reagent grade and used as such. Melting ranges were recorded with a Thomas-Hoover capillary melting point apparatus and are in Celsius. Infrared (IR) spectra were recorded on a Perkin-Elmer FT-IR spectrometer 1000. All solid sample spectra were recorded on their KBr pellets. NMR spectra were recorded on a JEOL Eclipse 400 spectrometer using CDCl₃ or DMSO-d₆ purchased from Cambridge Isotope Laboratories. Low and high resolution mass spectra (EI, CI and FAB) were obtained at Indiana University Mass Spectrometry Laboratory, Bloomington, Ind. Eastman silica gel and alumina strips with fluorescent indicator were used for thin-layer chromatography (TLC). Aziridine was prepared according to the procedure described by: Allen, C. F. H.; Spangler, F. W.; Webster, E. R. Organic Syntheses Collective Volume IV 1963, 433 and stored in refrigerator over KOH pellets.

### 7-Amino-2-formyl-6-methoxyguinoline-5,8-dione (49)

In a dry 25-mL round-bottomed, two-necked flask equipped with a magnetic bar and a water cooled reflux condenser under argon, 7-amino-6-methoxy-2-methylquinoline-5,8-dione (48, 100 mg, 0.46 mmol), selenium dioxide (91.7 mg, 0.83 mmol), 7 mL dried and distilled 1,4-dioxane, and 0.08 mL of water were stirred at reflux in an oil bath for 9 h. Another 0.2 equivalent of selenium dioxide (10.2 mg, 0.092 mmol) and water (0.001) was added to the mixture and stirred and refluxed for 3 more hours. The reaction was monitored by TLC. The mixture was hot filtered and washed with hot chloroform. The filtrate was rota-evaporated to dryness. The dried compound was dissolved in 100 mL of chloroform and washed with saturated sodium bicarbonate solution (3×15 mL) and then cold water (15 mL). The solution was dried over magnesium sulfate and rota-evaporated to dryness and then further dried under a vacuum pump. The dark red solid product weighed 54.6 mg (51%): ¹H NMR (CDCl₃) δ 10.25 (1H, s), 8.53 (1H, d, J=8.0 Hz), 8.21 (1H, d, J=8.0 Hz), 5.30 (2H, s), 4.10 (3H, s); IR (KBr) νₘₐₓ 3401, 3309, 1710, 1685, 1615, 1557, 1440, 1398, 1350, 1225, 1071, 1011, 919, 873, 744, 514 cm⁻¹; EIMS m/e (relative intensity) 232.1 (M+, 100.0), 220.1 (26.4), 219.1 (84.2), 218.1 (50.5), 205.1 (37.5), 204.1 (23.6), 191.1 (33.4), 189.1 (30.9), 106.1 (22.1); Anal. calcd. for C₁₁H₈N₂O₄; C, 56.90; H, 3.47; N, 12.06 found C, 56.73; H, 3.71; N, 11.88.

### 7-Amino-6-chloro-2-formylquinoline-5,8-dione (52)

In a dry 100 mL round-bottomed, two-necked flask equipped with a magnetic bar and a water cooled reflux condenser under argon, 7-amino-6-chloro-2-methylquinoline-5,8-dione (51, 133.8 mg, 0.6 mmol), selenium dioxide (106.6 mg), 20 mL dried and distilled 1,4-dioxane, and 0.09 mL of water were stirred at reflux in an oil bath for 23 h. The reaction was monitored by TLC. The mixture was hot filtered and the solid was washed with hot chloroform. The filtrate was rota-evaporated to dryness. The dried material was dissolved in 480 mL of chloroform and washed with saturated sodium chloride solution (4×60 mL). The solution was dried over magnesium sulfate and rota-evaporated to dryness and then further dried under a vacuum pump. The product weighed 105.2 mg (74%): ¹H NMR (CDCl₃) δ 10.30 (1H, s), 8.67 (1H, d, J=8.1 Hz), 8.28 (1H, d, J=8.1 Hz), 5.60 (2H, br s); IR (KBr) νₘₐₓ 3434, 3292, 3057, 2924, 1712, 1698, 1633, 1602, 1551, 1381, 1350, 1326, 1257, 1075, 831, 742 cm⁻¹; EIMS m/e 238.0, 236.0 (M+), 222.0, 210.0, 208.0, 201.0, 187.0, 171.0.

### 6-Methoxydemethyllavendamycin Methyl Ester (30)

In a dry 250-mL 3-necked, round-bottomed flask equipped with a Dean-Stark trap under an argon flow, 7-amino-2-formyl-6-methoxyquinoline-5,8-dione (49, 48 mg, 0.2 mmol) and tryptophan methyl ester (44 mg, 0.2 mmol) were dissolved in dry anisole (120 mL). The reaction mixture was heated to reflux gradually over 3 h and then refluxed for another 8 h. The reaction mixture was cooled and rota-evaporated to dryness. The solid was washed with acetone (20 mL), filtered, and air dried. The dark orange brown solid weighed 38.4 mg (45%): mp>280° C.; ¹H NMR (CDCl₃) δ 11.94 (1H, br s), 9.11 (1H, d, J=8.3 Hz), 8.98 (1H, s), 8.53 (1H, d, J=8.3 Hz), 8.23 (1H, d, J=7.4 Hz), 7.78 (1H, d, J=8.1 Hz), 7.65 (1H, m), 7.38 (1H, m), 5.20 (2H, br s), 4.12 (3H, s), 4.09 (3H, s); IR (KBr) νₘₐₓ 3444, 3335, 1712, 1680, 1615, 1557, 1489, 1435, 1400, 1346, 1264, 1232, 1121, 1075, 1017, 921, 735 cm⁻¹; FABMS m/e (relative intensity) 431 (M+1, 10), 309 (70), 275 (20), 195 (22), 155 (73), 135 (59), 119 (100); HRMS calcd. for C₂₃H₁₉N₄O₅ 431.135 (M+3) found 431.135.

### 6-Methoxylavendamycin Methyl Ester (31)

In a dry 100-mL 3-necked, round-bottomed flask equipped with a Dean-Stark trap under an argon flow, 7-amino-2-formyl-6-methoxyquinoline-5,8-dione (49, 23 mg, 0.1 mmol) and β-methyltryptophan methyl ester (23 mg, 0.1 mmol) were dissolved in dry anisole (60 mL). The reaction mixture was heated to reflux gradually over 3 h and then refluxed for another 7 h. The reaction mixture was cooled and rota-evaporated to dryness. The solid was washed with acetone (3 mL), filtered, and air dried. The orange brown solid weighed 18 mg (41%): mp>280° C.; ¹H NMR (CDCl₃) δ 11.99 (1H, br s), 9.03 (1H, d, J=8.3 Hz), 8.49 (1H, d, J=8.3 Hz), 8.35 (1H, d, J=7.8 Hz), 7.80 (1H, d, J=8.0 Hz), 7.64 (1H, m), 7.38 (1H, m), 5.18 (2H, br s), 4.11 (3H, s), 4.06 (3H, s), 3.20 (3H, s); IR (KBr) νₘₐₓ3325, 1701, 1616, 1489, 1437, 1400, 1337, 1276, 1234, 1079, 918, 733 cm⁻¹; EIMS m/e (relative intensity) 442.13 (M+, 90.1), 374.01 (100), 316.98 (22.4), 201.00 (22.2), 151.00 (58.6); HRMS calcd. for C₂₄H₁₈N₄O₅ 442.127 found 442.127.

### 6-Methoxydemethyllavendamycin Amide (32)

In a dry 250-mL 3-necked, round-bottomed flask equipped with a Dean-Stark trap under an argon flow, 7-amino-2-formyl-6-methoxyquinoline-5,8-dione (49, 46.5 mg, 0.2 mmol) and tryptophanamide (42 mg, 0.2 mmol) were dissolved in dry anisole (120 mL). The reaction mixture was heated to reflux gradually over 3 h and then refluxed for another 9 h. The solution was cooled and rota-evaporated to dryness. The solid was washed with acetone (20 mL), filtered, and air dried. The tan solid weighed 27 mg (32%): ¹H NMR (DMSO-d₆) δ 11.99 (1H, br s), 9.41 (1H, d, J=8.1 Hz), 9.07 (1H, s), 8.60 (1H, br s), 8.53 (1H, d, J=8.1 Hz), 8.47 (1H, d, J=8.0 Hz), 7.78 (1H, d, J=8.0 Hz), 7.74-7.70 (1H, m), 7.67 (1H, br s), 7.44-7.40 (1H, m), 7.14 (2H, br s), 3.89 (3H, s); EIMS m/e (relative intensity) 413.2 (M+, 100), 369.2 (13), 353.1 (12), 325.1 (19), 270.1 (10), 242.1 (15), 130.1 (36); HRMS calcd. for C₂₂H₁₅N₅O₄ 413.116 found 413.112.

### 6-Chlorolavendamycin Methyl Ester (33)

In a dry 500-mL 3-necked, round-bottomed flask equipped with a magnetic stirring bar, and a Dean-Stark trap under an argon flow, 7-amino-6-chloro-2-formylquinoline-5,8-dione (52, 118.5 mg, 0.5 mmol) was placed with β-methyltryptophan methyl ester (115 mg, 0.5 mmol) in 300 mL of dry anisole. The solution was stirred and heated slowly to 130° C. over 3 h. The reaction mixture was refluxed for one hour. The mixture was then cooled down to room temperature under argon. The solvent was rota-evaporated to dryness and then the solid was washed with acetone (20 mL). The resulting solid was vacuum filtered and dried under a vacuum pump. The product weighed 131.9 mg (59%): ¹H NMR (CDCl₃) δ 11.89 (1H, br s), 9.10 (1H, d, J=8.4 Hz), 8.61 (1H, d, J=8.4 Hz), 8.38 (1H, d, J=7.7 Hz), 7.80 (1H, d, J=8.0 Hz), 7.67 (1H, m), 7.41 (1H, m), 5.70 (2H, br s), 4.08 (3H, s), 3.22 (3H, s); CIMS m/e (relative intensity) 446.1 (M+, 100), 416.0 (10.3), 386.0 (64.4), 351.1 (22.0), 113.0 (9.0); HRMS calcd. for C₂₃H₁₅N₄O₄Cl 446.081 found 446.078.

### 6-Chlorodemethyllavendamycin Ethyl Ester (34)

In a dry 500-mL 3-necked, round-bottomed flask equipped with a magnetic stirring bar and a Dean-Stark trap under an argon flow, 7-amino-6-chloro-2-formylquinoline-5,8-dione (52, 47.4 mg, 0.2 mmol) was placed with tryptophan ethyl ester (46.4 mg, 0.2 mmol) in 120 mL of dry anisole. The solution was stirred and heated slowly to 130° C. over 3 h. The reaction mixture was refluxed for one hour. The mixture was then cooled down to room temperature under argon. The solvent was rota-evaporated to dryness and then the solid was washed with acetone (20 mL). The resulting solid was vacuum filtered and dried under a vacuum pump. The product weighed 47 mg (49%): ¹H NMR (CDCl₃) δ 11.86 (1H, br s), 9.22 (1H, d, J=8.3 Hz), 8.99 (1H, s), 8.67 (1H, d, J=8.3 Hz), 8.27 (1H, d, J=7.8 Hz), 7.77 (1H, d, J=7.3 Hz), 7.68 (1H, m), 7.42 (1H, m), 5.60 (2H, br s), 4.57 (2H, q, J=7.2 Hz), 1.57 (3H, t, J=7.2 Hz); IR (KBr) νₘₐₓ 3463, 3312, 2976, 1708, 1611, 1367, 1330, 1229, 826, 740 cm⁻¹; FABMS m/e (relative intensity) 447 (M+, 5.2), 391 (36.7), 371 (37.4), 307 (19.9), 289 (14.5), 167 (11.7), 154 (100), 149 (45.5), 136 (68.6), 129 (31.1), 106 (24.3); HRMS calcd. for C₂₃H₁₅N₄O₄Cl 447.086 (M+1) found 447.086.

### 6-Chlorodemethyllavendamycin Pyrrolidine Amide (35)

In a dry 250-mL 3-necked, round-bottomed flask equipped with a magnetic stirring bar and a Dean-Stark trap under an argon flow, 7-amino-6-chloro-2-formylquinoline-5,8-dione (52, 35.5 mg, 0.15 mmol) was placed with tryptophan pyrrolidine amide (38.6 mg, 0.15 mmol) in 60 mL of dry anisole. The solution was stirred and heated slowly to 110° C. over 3.5 h. The reaction mixture was refluxed for 30 minutes. The mixture was then cooled down to room temperature under argon. The solvent was rota-evaporated to dryness and then the solid was washed with petroleum ether (10 mL). The resulting solid was vacuum filtered and dried under a vacuum pump. Flash column chromatography (silica gel 40, 6 in., d=1.8 cm, 2% MeOH in CH₂Cl₂ as eluent) of the crude product afforded 23.7 mg of an orange solid (36%): ¹H NMR (DMSO-d₆) δ 11.82 (1H, br s), 8.86 (1H, d, J=8.4 Hz), 8.84 (1H, s), 8.55 (1H, d, J=8.4 Hz), 8.48 (1H, d, J=7.7 Hz), 7.74 (1H, d, J=8.4 Hz), 7.70 (1H, m), 7.39 (1H, m), 3.97 (2H, m), 3.64 (2H, m), 1.95 (4H, m), EIMS m/e (relative intensity) 471.1 (M+, 33), 376.1 (33), 374.1 (100), 236 (36), 181 (26), 169 (29), 119 (32), 97 (21).

### 6-Chlorodemethyllavendamycin Isoamyl Ester (36)

In a dry 500-mL 3-necked, round-bottomed flask equipped with a magnetic stirring bar and a Dean-Stark trap under an argon flow, 7-amino-6-chloro-2-formylquinoline-5,8-dione (52, 47.4 mg, 0.2 mmol) was placed with tryptophan isoamyl ester (56 mg, 0.2 mmol) in 120 mL of dry anisole. The solution was stirred and heated slowly to 130° C. over 3 h. The reaction mixture was refluxed for one hour. The mixture was then cooled down to room temperature under argon. The solvent was rota-evaporated to dryness and then the solid was washed with acetone (20 mL). The resulting solid was vacuum filtered and dried under a vacuum pump. The product weighed 54.5 mg (56%): ¹H NMR (CDCl₃) δ 11.91 (1H, br s), 9.20 (1H, d, J=8.2 Hz), 9.00 (1H, s), 8.65 (1H, d, J=8.2 Hz), 8.25 (1H, d, J=8.1 Hz), 7.81-7.60 (2H, m), 7.45 (1H, m), 5.70 (2H, br s), 4.50 (2H, t, J=6.9 Hz), 1.90 (2H, m), 1.26 (1H, m), 1.08 (6H, d, J=8.1 Hz); IR (KBr) νₘₐₓ 3462, 3337, 2957, 1696, 1614, 1473, 1330, 1262, 1224, 828, 738 cm⁻¹; EIMS m/e (relative intensity) 488 (M+, 40.8), 454 (24.1), 446 (25.1), 376 (35.7), 375 (29.9), 374 (96.0), 340 (46.4), 199 (100.0), 184 (35.7), 91 (27.2), 69 (32.1), 57 (37.7); HRMS calcd. for C₂₆H₂₁N₄O₄Cl 488.125 found 488.125.

### 6-Chlorodemethyllavendamycin Octyl Ester (37)

In a dry 500-mL 3-necked, round-bottomed flask equipped with a magnetic stirring bar and a Dean-Stark trap under an argon flow, 7-amino-6-chloro-2-formylquinoline-5,8-dione (52, 47.4 mg, 0.2 mmol) was placed with tryptophan octyl ester (63.2 mg, 0.2 mmol) in 120 mL of dry anisole. The solution was stirred and heated slowly to 130° C. over 3 h. The reaction mixture was refluxed for 2 hours. The mixture was then cooled down to room temperature under argon. The solvent was rota-evaporated to dryness and then the solid was washed with acetone (20 mL). The resulting solid was vacuum filtered and dried under a vacuum pump. The product weighed 27.3 mg (26%): ¹H NMR (CDCl₃) δ 11.82 (1H, br s), 9.18 (1H, d, J=8.3 Hz), 8.96 (1H, s), 8.63 (1H, d, J=8.3 Hz), 8.25 (1H, d, J=7.8 Hz), 7.75 (1H, d, J=7.4 Hz), 7.67 (1H, m), 7.39 (1H, m), 5.60 (2H, br s), 4.48 (2H, t, J=6.9 Hz), 1.90 (2H, m), 1.27 (10H, m), 0.88 (3H, t, J=3.8 Hz); IR (KBr) νₘₐₓ 3470, 3332, 2925, 1725, 1611, 1472, 1329, 1228, 830, 739 cm⁻¹; EIMS m/e (relative intensity) 532 (M+2, 3.1), 446 (29.2), 265 (22.0), 250 (27.7), 226 (23.5), 212 (22.6), 199 (100.0), 184 (40.3), 149 (33.8), 97 (24.0), 91 (30.6), 83 (32.8), 69 (50.2); HRMS calcd. for C₂₉H₂₇N₄O₄Cl 530.172 found 530.171.

### 6-Chlorodemethyllavendamycin Amide (38)

In a dry 500-mL 3-necked, round-bottomed flask equipped with a magnetic stirring bar and a Dean-Stark trap under an argon flow, 7-amino-6-chloro-2-formylquinoline-5,8-dione (52, 47.4 mg, 0.2 mmol) was placed with tryptophanamide (40.8 mg, 0.2 mmol) in 120 mL of dry anisole. The solution was stirred and heated slowly to 130° C. over 3 h. The reaction mixture was refluxed for 13 h. The mixture was then cooled down to room temperature under argon. The solvent was rota-evaporated to dryness and then the solid was washed with acetone (20 mL). The resulting solid was vacuum filtered and dried under a vacuum pump. The product weighed 44.4 mg (53%): ¹H NMR (DMSO-d₆) δ 11.95 (1H, br s), 10.21 (2H, s), 9.44 (1H, d, J=8.4 Hz), 9.07 (1H, s), 8.52 (1H, d, J=8.4 Hz), 8.49 (1H, d, J=8.4 Hz), 7.90 (2H, br s), 7.79-7.67 (2H, m), 7.43 (1H, m); IR (KBr) νₘₐₓ 3450, 3326, 1609, 1391, 1337, 1232, 830, 739 cm⁻¹; FABMS m/e (relative intensity) 420 (M+3, 1.6), 385 (9.9), 309 (9.1), 233 (35.7), 195 (11.6), 159 (14.4), 157 (100.0), 155 (33.5), 135 (32.2), 118 (74.0); HRMS calcd. for C₂₁H₁₅N₅O₃Cl 420.086 (M+3) found 420.086.

### 7-N-Acetyl-6-aziridinolavendamycin Methyl Ester (39)

To a stirred solution of 7-acetyllavendamycin methyl ester (11, 50 mg, 0.11 mmol) in dry chloroform (30 ml) was added aziridine (0.7 mL, 580 mg, 13.5 mmol) and the resulting orange solution was allowed to stir at room temperature for 24 h. The reaction mixture was rota-evaporated to dryness. The solid was washed with ether (10 mL) and further dried under a vacuum pump to afford 43 mg (79%) of the product as a yellowish orange powder: mp 250° C. (dec.); ¹H NMR (DMSO-d₆) δ 12.11 (1H, s), 9.57 (1H, s), 8.82 (1H, d, J=7.3 Hz), 8.57 (1H, d, J=7.3 Hz), 8.42 (1H, d, J=7.9 Hz), 7.83 (1H, d, J=7.9 Hz), 7.75-7.68 (1H, m), 7.47-7.40 (1H, m), 3.98 (3H, s), 3.10 (3H, s), 2.39 (4H, s), 2.17 (3H, s); IR (KBr) νₘₐₓ 3341, 3244, 3000, 2955, 1708, 1664, 1581, 1494, 1368, 1335, 1299, 1272, 1076, 736 cm⁻¹; EIMS m/e (relative intensity) 495.15 (M+, 100.0), 421.1 (28.2), 393.1 (87.2), 378.1 (25.8), 282.1 (10.2), 196.6 (10.2), 125.1 (10.8), 111.1 (14.4), 83.1 (19.7); HRMS calcd. for C₂₇H₂₁N₅O₅ 495.158 found 495.154.

### 7-N-Acetyl-6-pyrrolidinolavendamycin Methyl Ester (40)

To a stirred solution of 7-acetyllavendamycin methyl ester (11, 50 mg, 0.11 mmol) in dry chloroform (30 ml) was added pyrrolidine (0.2 mL, 170 mg, 2.4 mmol) and the resulting red-brown solution was allowed to stir at room temperature for 2 h. Then the reaction mixture was rota-evaporated to dryness. The solid was washed with ether (10 mL) and further dried under a vacuum pump to afford 51 mg (88%) of the product as a reddish brown powder: mp 210° C. (dec.); ¹H NMR (DMSO-d₆) δ 12.36 (1H, s), 9.33 (1H, s), 8.78 (1H, d, J=8.2 Hz), 8.50 (1H, d, J=8.2 Hz), 8.45 (1H, d, J=7.9 Hz), 7.82 (1H, d, J=7.9 Hz), 7.76-7.70 (1H, m), 7.48-7.41 (1H, m), 3.98 (3H, s), 3.78-3.58 (4H, br m), 3.11 (3H, s), 2.11 (3H, s), 1.95-1.75 (4H, br m); IR (KBr) νₘₐₓ 3437, 3326, 2948, 1712, 1664, 1590, 1553, 1398, 1331, 1294, 1276, 1073, 744 cm⁻¹; FABMS m/e (relative intensity) 546.2 (M+Na, 15.4), 527.3 (14.2), 507.3 (25.2), 506.3 (100.0), 413.5 (29.0), 391.5 (30.5), 232.0 (31.0), 176.0 (50.0), 154.0 (99.5); HRMS calcd. for C₂₉H₂₅N₅O₅Na (M+Na) 523.180 found 546.175.

### 7-N-Acetyl-6-aziridinodemethyllavendamycin Amide (41)

To a stirred solution of 7-acetyldemethyllavendamycin amide (16, 50 mg, 0.12 mmol) in dry chloroform (25 ml) and dry ethanol (25 ml) was added aziridine (0.7 mL, 580 mg, 13.5 mmol) and the resulting reddish brown solution was allowed to stir at room temperature for 48 h. Then the reaction mixture was rota-evaporated to dryness. The solid was washed with ether (10 mL) and further dried under a vacuum pump to afford 52 mg (93%) of the product as a reddish brown powder: mp>260° C.; ¹H NMR (DMSO-d₆) δ 12.05 (1H, s), 9.57 (1H, s), 9.45 (1H, d, J=8.2 Hz), 9.07 (1H, s), 8.62 (1H, s), 8.55-8.49 (2H, m), 7.83 (1H, d, J=8.2 Hz), 7.74-7.69 (1H, m), 7.68 (1H, s), 7.44-7.38 (1H, m), 2.39 (4H, s), 2.17 (3H, s); IR (KBr) νₘₐₓ 3429, 3326, 1675, 1653, 1577, 1494, 1472, 1387, 1368, 1303, 1231, 736 cm⁻¹; ESMS m/e (relative intensity) 468 (M+2, 2.50), 467 (100), 441 (60), 318 (30), 296 (43); HRMS calcd. for C₂₅H₁₉N₆O₄ (M+1) 467.150 found 467.148.

### 7-N-Acetyl-6-pyrrolidinodemethyllavendamycin Amide (42)

To a stirred solution of 7-acetyldemethyllavendamycin amide (16, 50 mg, 0.12 mmol) in DMF (10 mL) was added pyrrolidine (0.3 mL, 255 mg, 3.6 mmol) and the resulting dark brown solution was allowed to stir at room temperature for 2 h. Then the reaction mixture was rota-evaporated to dryness. The solid was washed with THF (10 mL) and further dried under a vacuum pump to afford 50 mg (86%) of the product as a reddish brown powder: mp 245° C. (dec.); ¹H NMR (DMSO-d₆) δ 12.25 (1H, s), 9.40-9.30 (2H, m), 9.06 (1H, s), 8.61 (1H, s), 8.52 (1H, d, J=7.7 Hz), 8.43 (1H, d, J=8.2 Hz), 7.78 (1H, d, J=7.9 Hz), 7.73-7.69 (1H, m), 7.67 (1H, s), 7.43-7.38 (1H, m), 3.78-3.60 (4H, br m), 2.11 (3H, s), 1.98-1.75 (4H, br m); IR (KBr) νₘₐₓ 3452, 3296, 2970, 2926, 1675, 1623, 1590, 1533, 1390, 1301, 1235, 747 cm⁻¹; FABMS m/e (relative intensity) 495.2 (M+1, 9.9), 484.4 (26.2), 483.4 (100.0); HRMS calcd. for C₂₇H₂₃N₆O₄ 495.182 (M+1) found 495.178.

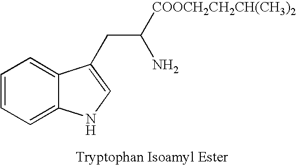

Isoamyl alcohol was dried over anhydrous cupric sulfate for 24 h and then distilled under argon. Tryptophan (1.43 g, 7 mmol) was placed in a 100-mL round-bottomed flask along with 60 mL of the dried isoamyl alcohol and 10 mL of HCl/ether solution. The solution was refluxed in an oil bath for 22 h. The mixture was then rota-evaporated to dryness. A portion of the resulting tryptophan isoamyl ester hydrochloride (723 mg, 2.84 mmol) was suspended in 36 mL ethyl acetate. To this stirred suspension, a 14% solution of ammonium hydroxide (−3 mL) was added until the aqueous layer was at pH=8. The aqueous layer was separated and the organic layer was washed with a saturated sodium chloride solution (3×2 mL) and water (2 mL) and dried over magnesium sulfate. The solution was filtered and rota-evaporated to dryness. The thick liquid was further dried under a vacuum pump at 50-60° C. for two days. The overall product weighed 1.6 g (83%): mp 50° C. (dec.); ¹H NMR (CDCl₃) δ 8.08 (1H, br s), 7.59 (1H, d, J=7.3 Hz), 7.33 (1H, d, J=7.6 Hz), 7.20 (1H, m), 7.10 (1H, m), 7.00 (1H, s), 4.10 (2H, t, J=6.7 Hz), 3.81 (1H, m), 3.22-2.99 (2H, m), 1.60 (4H, m), 1.50 (1H, m), 0.90 (6H, d, J=6.3 Hz); IR (KBr) νₘₐₓ 3353, 3058, 2957, 2928, 2870, 1727, 1670, 1620, 1519, 1458, 1436, 1341, 1234, 1204, 1098, 1010, 943, 743 cm⁻¹; CIMS m/e (relative intensity) 275.2 (M+1, 20), 274.2 (28), 257.1 (37), 159.1 (10), 131 (22), 130.1 (100); HRMS calcd. for C₁₆H₂₂N₂O₂ 274.168 found 274.168.

The foregoing detailed description and accompanying drawings have been provided by way of explanation and illustration, and are not intended to limit the scope of the appended claims. Many variations in the presently preferred embodiments illustrated herein will be obvious to one of ordinary skill in the art, and remain within the scope of the appended claims and their equivalents.

1. A compound having a formula: 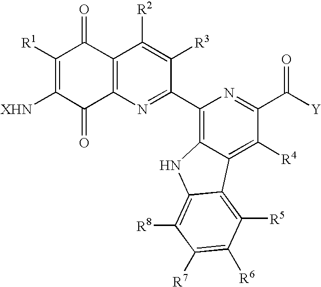
or a pharmaceutically acceptable salt thereof, wherein:
X is hydrogen or R¹⁰(O)—;
Y is hydroxy, alkoxy, amino, Ph(CH₂)ₘO—, HO(CH₂)ₙO— or —O(CH₂)ₒOPO₃H₂, wherein m, n, and o are each independently 1, 2, 3, 4, 5, 6, 7, 8, 9 or 10;
R¹ is hydrogen, amino, alkoxy, halo, acyl, aziridinyl, pyrrolidinyl, piperidinyl or pyridinyl;
R², R³, R⁴, R⁵, R⁶, R⁷, and R⁸ are each independently hydrogen or a substituted or unsubstituted radical selected from the group consisting of alkyl, aryl, cycloalkyl, alkenyl, alkynyl, heteroalkyl, heterocyclic, heteroalkenyl, heteroalkynyl, halo, nitro, cyano, alkoxy, amino, amido, thioamido, acyl, thioacyl, and imino; and
R¹⁰ is hydrogen or a substituted or unsubstituted radical selected from the group consisting of alkyl, aryl, cycloalkyl, alkenyl, alkynyl, heteroalkyl, heterocyclic, heteroalkenyl, and heteroalkynyl;
with a proviso that when R¹ is hydrogen, halo, amino, alkoxy or acyl, then Y is not hydroxy, alkoxy or amino.

2. The invention of claim 1 wherein each of R², R³, R⁵, R⁶, R⁷, and R⁸ is hydrogen.

3. The invention of claim 2 wherein R⁴ is hydrogen or alkyl.

4. The invention of claim 3 wherein R⁴ is hydrogen or methyl.

5. The invention of claim 4 wherein Y is —O-n-C₄H₉, —O-n-C₈H₁₇, Ph(CH₂)ₘO—, HO(CH₂)ₙO— or —O(CH₂)ₒOPO₃H₂.

6. The invention of claim 5 wherein Y is Ph(CH₂)ₘO—, HO(CH₂)ₙO— or —O(CH₂)ₒOPO₃H₂.

7. The invention of claim 6 wherein Y is HO(CH₂)ₙO— or —O(CH₂)ₒOPO₃H₂, and wherein n and o are independently 1, 2, 3, 4, or 5.

8. The invention of claim 7 wherein n and o are independently 1, 2 or 3.

9. The invention of claim 8 wherein n and o are 2.

10. The invention of claim 9 wherein R¹ is hydrogen.

11. The invention of claim 4 wherein R¹ is amino, alkoxy, halo, acyl, aziridinyl, pyrrolidinyl piperidinyl or pyridinyl.

12. The invention of claim 11 wherein R¹ is R¹¹NH—, R¹¹R¹²N—, methoxy, chloro, aziridinyl, pyrrolidinyl or piperidinyl, and wherein R¹¹ and R¹² are each independently a substituted or unsubstituted radical selected from the group consisting of alkyl, aryl, cycloalkyl, alkenyl, alkynyl, heteroalkyl, heterocyclic, heteroalkenyl, and heteroalkynyl.

13. The invention of claim 12 wherein Y is a substituted or unsubstituted radical selected from the group consisting of alkoxy, amino, and heterocyclic.

14. The invention of claim 13 wherein Y is —OCH₃, —NH₂, —OCH₂CH₃, 1-pyrrolidinyl, —O-iso-amyl or —O-n-octyl.

15. A method of synthesizing a compound having a formula: 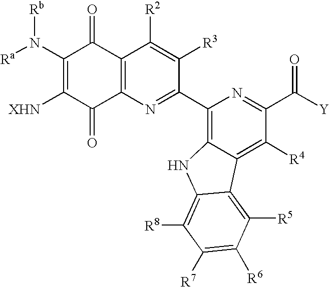
wherein:
X is hydrogen or R¹⁰C(O)—;
Y is hydroxy, alkoxy or amino;
R^{a} and R^{b} are either (a) independently hydrogen or a substituted or unsubstituted radical selected from the group consisting of alkyl, aryl, cycloalkyl, alkenyl, alkynyl, heteroalkyl, heterocyclic, heteroalkenyl, and heteroalkynyl or (b) together with the nitrogen atom to which they are attached form a three-, four-, five-, six-, seven- or eight-membered cyclic ring;
R¹ is amino, aziridinyl, pyrrolidinyl, piperidinyl or pyridinyl;
R², R³, R⁴, R⁵, R⁶, R⁷, and R⁸ are each independently hydrogen or a substituted or unsubstituted radical selected from the group consisting of alkyl, aryl, cycloalkyl, alkenyl, alkynyl, heteroalkyl, heterocyclic, heteroalkenyl, heteroalkynyl, halo, nitro, cyano, alkoxy, amino, amido, thioamido, acyl, thioacyl, and imino; and
R¹⁰ is hydrogen or a substituted or unsubstituted radical selected from the group consisting of alkyl, aryl, cycloalkyl, alkenyl, alkynyl, heteroalkyl, heterocyclic, heteroalkenyl, and heteroalkynyl;
the method comprising:
reacting a compound having a structure: 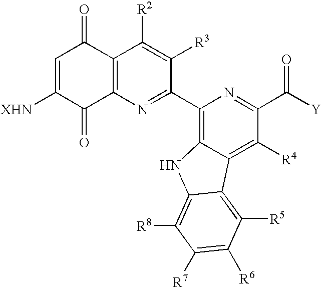
with a compound having a structure:

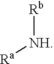

16. The invention of claim 15 wherein each of R², R³, R⁵, R⁶, R⁷, and R⁸ is hydrogen.

17. The invention of claim 16 wherein R⁴ is hydrogen or alkyl.

18. The invention of claim 17 wherein R⁴ is hydrogen or methyl.

19. A method of treating cancer comprising administering to an animal in need thereof a therapeutically effective amount of the compound of claim 1, 11, 12, 13 or 14.

20. A method of treating HIV infection comprising administering to an animal in need thereof a therapeutically effective amount of the compound of claim 1, 5, 6, 7, 8, 9 or 10.

21. The invention of claim 20 further comprising administering a therapeutically effective amount of a compound selected from the group consisting of HIV-RT, AZT, ddI, d4T, 3TC, ddc, ABC, and combinations thereof.
